(19) United States
(12) Patent Application Publication
(10) Pub. No.: US 2015/0272992 A1
(43) Pub. Date: Oct. 1, 2015

(54) Treatment of Tumors with Activated Mesenchymal Stem Cells

(71) Applicants: THE TEXAS A&M UNIVERSITY SYSTEM, College Station, TX (US); TEMPLE THERAPEUTICS, INC., Framingham, MA (US)

(72) Inventors: Darwin J. Prockop, Philadelphia, PA (US); Ryang Hwa Lee, Temple, TX (US); Nara Yoon, Temple, TX (US); John Reneau, Rochester, MN (US); Barry Berkowitz, Framingham, MA (US)

(21) Appl. No.: 14/436,637

(22) PCT Filed: Oct. 17, 2013
(86) PCT No.: PCT/US2013/065350

§ 371 Date: Apr. 17, 2015

Related U.S. Application Data
(60) Provisional application No. 61/717,682, filed on Oct. 24, 2012.

(51) Int. Cl. A61K 35/28; A61K 31/704

(52) U.S. Cl. CPC A61K 35/28; A61K 31/704

(57) ABSTRACT

A method of treating a tumor in an animal by administering to the animal mesenchymal stem cells that have been contacted with a stimulating, or activating agent, such as TNF-a, that stimulates the mesenchymal stem cells to express increased amounts of at least one agent, such as TRAIL and/or DKK-3, that inhibits, prevents, or destroys the growth of tumors. The stimulated, or activated mesenchymal stem cells may be administered in combination with at least one chemotherapeutic agent, such as doxorubicin.

This application claims priority based on provisional Application Ser. No. 61/717,682, filed Oct. 24, 2012, the contents of which are incorporated by reference in their entirety.

This invention relates to the treatment of tumors with mesenchymal stem cells. More particularly, this invention relates to treating tumors in an animal by administering to an animal mesenchymal stem cells that have been contacted with at least one agent that activates or stimulates the mesenchymal stem cells to express increased amounts of at least one agent that inhibits, prevents, or destroys the growth of tumors.

This invention also relates to the treatment of tumors in an animal by administering to the animal mesenchymal stem cells in combination with at least one chemotherapeutic agent.

Non-hematopoietic progenitor cells derived from bone marrow, known as mesenchymal stem cells or multipotent stromal cells (MSCs), have been investigated for the treatment of cancers because they are able to home preferentially to tumors and incorporate into tumor stroma (Kanehira et al Cancer Gene Therapy, Vol. 14, pgs. 894-903 (2007); Kucerova et al., Cancer Res., Vol. 67, pgs. 6304-6313 (2007); Studeny et al., J. Nat. Cancer Inst., Vol. 96, pgs. 1593-1603 (2004); Xin et al. Stem Cells, Vol. 25, pgs. 1618-1626, (2007)), but previous research has yielded conflicting results. Some reports showed that MSCs inhibited tumor growth (Djouad et al., Transplantation, Vol. 82, pgs. 1060-1066 (2006); Kidd, et al., Cytotherapy, Vol. 12, pgs. 615-625 (2010); Tian et al., 2010; Zhu et al., 2009), but others reported that the MSCs promoted tumor growth or metastases (Djouad, et al., Blood, Vol. 102, pgs. 3837-3844 (2003); Karnoub, et al., Nature, Vol. 449, pgs. 559-563 (2007); Kurtova, et al., Blood, Vol. 114, pgs. 4441-4450 (2009); Patel, et al., J. Immunol., Vol. 184, pgs. 5885-5894 (2010)). Recently, it was observed that incubation of human MSCs (hMSCs) with recombinant human tumor necrosis factor-α (TNF-α) activated the cells to express a number of potentially therapeutic proteins including tumor necrosis factor-α related apoptosis inducing ligand (TRAIL) (Rahman et al., Breast Cancer Research Treat., Vol. 113, pgs. 217-230 (2009)). TRAIL causes apoptosis in many malignant cells but not in normal cells; for this reason, soluble recombinant TRAIL was employed in a series of clinical trials (Gazitt, Leukemia, Vol. 13, pgs. 1817-1824 (1999); Johnstone, et al., Nat. Rev. Cancer, Vol. 8, pgs. 782-798 (2008); Kelley, et al., J. Pharmacol. Exp. Ther., (2001)), but the success was limited by the short half-life in serum (Kelley et al., 2001) and the lower bioactivity of the soluble protein compared to the membrane bound form (Rus et al., Clin. Immunol., Vol. 117, pgs. 48-56 (2005)). One strategy to overcome the limitations of soluble TRAIL is to use hMSCs as delivery vectors and thereby capitalize on their ability to home to tumors. hMSCs that were transduced with viral vectors to over-express TRAIL suppressed tumor xenografts in several in vivo models including glioma, colorectal carcinoma, and metastatic breast cancer (Grisendi et al., Cancer Res., Vol. 70, pgs. 3718-3729 (2010); Loebinger et al., Cancer Res., Vol. 69, pgs. 4134-4142 (2009); Menon et al., Stem Cells, Vol. 27, pgs. 2320-2330 (2009); Mohr et al., J. Cell Mol. Med., Vol. 12, pgs. 2628-2643 (2008); Mueller et al., Cancer Gene Therapy, Vol. 18, pgs 229-239 (2011)). The use of viral vectors, however, introduces limitations such as insertional mutagenesis and phenotypic changes in the hMSCs.

It also was observed that DKK-3 expression was increased upon exposure of hMSCs to TNF-α. DKK-3 is suppressed in many breast cancer cell lines because the gene promoter is hypermethylated (Kuphal, et al., Oncogene, Vol. 25, pgs. 5027-5036 (2006)), an observation suggesting DKK-3 is a tumor suppressor gene. Furthermore, several reports showed that epigenetic inactivation of DKK-3 stimulates the Wnt/β-catenin pathway that plays an important role in tumorigenesis (Bafico et al., Cancer Cell, Vol. 6, pgs. 497-506 (2004); Clevers, Cell, Vol. 127, pgs. 469-480 (2006); Vogelstein and Kinzler, Nat. Med., Vol. 10, pgs. 789-799 (2004)). This inactivation promotes the growth of human breast, lung, and cervical cancer (Lee et al., Int. J. Cancer, Vol. 124, pgs. 287-297 (2009); Veeck et al., Breast Cancer Res., Vol. 10, pg. R82 (2008); Yue et al., Carcinogenesis, Vol. 29, pgs. 84-92 (2008).

Because hMSCs activated with TNF-α expressed both TRAIL and DKK-3, the hypothesis that activated hMSCs are tumor suppressive was tested. Applicants have shown that pre-activated hMSCs reduced the tumor burden in a lung metastatic xenograft model that was produced with MDA-MB-231 (MDA) in vivo. They also induced apoptosis of MDA cells and several other TRAIL-sensitive cancer cell lines and prevented cell cycle progression of MDA cells in vitro.

Thus, in accordance with an aspect of the present invention, there is provided a method of treating a tumor in an animal. The method comprises administering to the animal mesenchymal stem cells which have been contacted with an agent that stimulates, or activates, the mesenchymal stem cells to express increased amounts of at least one agent that inhibits, prevents, or destroys the growth of tumors. The mesenchymal stem cells are administered in an amount effective to inhibit, prevent, or destroy the growth of a tumor in an animal.

The term “increased amounts of at least one agent that inhibits, prevents, or destroys the growth of the tumors,” as used herein, means that the mesenchymal stem cells produce or express more of the agent that inhibits, prevents, or destroys the growth of tumors after being contacted with the agent that stimulates or activates the mesenchymal stem cells, than prior to being contacted with the agent that stimulates or activates the mesenchymal stem cells.

In a non-limiting embodiment, the mesenchymal stem cells are contacted with the agent that stimulates the mesenchymal stem cells to express increased amounts of the agent that inhibits, prevents, or destroys the growth of tumors prior to being administered to the animal. In another non-limiting embodiment, the mesenchymal stem cells are contacted with the agent that stimulates the mesenchymal stem cells to express increased amounts of the agent that inhibits, prevents, or destroys the growth of tumors concurrently with the administration of the mesenchymal stem cells to the animal.

The mesenchymal stem cells may be administered to any animal. Such animals include mammals, including human and non-human primates, birds, reptiles, amphibians and fish.

The MSCs can be obtained from any source. The MSCs may be autologous with respect to the recipient (obtained from the same host) or allogeneic with respect to the recipient. In addition, the MSCs may be xenogeneic to the recipient (obtained from an animal of a different species); for example, rat MSCs may be used to treat a tumor in a human.

In a further non-limiting embodiment, MSCs used in the present invention can be isolated, from the bone marrow of any species of mammal, including but not limited to, human, mouse, rat, ape, gibbon, bovine. In a non-limiting embodiment, the MSCs are isolated from a human, a mouse, or a rat. In another non-limiting embodiment, the MSCs are isolated from a human.

Any medium capable of supporting MSCs in vitro may be used to culture the MSCs. Media formulations that can support the growth of MSCs include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, 0 to 20% fetal bovine serum (FBS) or 1-20% horse serum is added to the above medium in order to support the growth of MSCs. A defined medium, however, also can be used if the growth factors, cytokines, and hormones necessary for culturing MSCs are provided at appropriate concentrations in the medium. Media useful in the methods of the invention may contain one or more compounds of interest, including but not limited to antibiotics, mitogenic or differentiation compounds useful for the culturing of MSCs. The cells may be grown in one non-limiting embodiment, at temperatures between 27° C. to 40° C., in another non-limiting embodiment at 31° C. to 37° C., and in another non-limiting embodiment in a humidified incubator. The carbon dioxide content may be maintained between 2% to 10% and the oxygen content may be maintained between 1% and 22%; however, the invention should in no way be construed to be limited to any one method of isolating and culturing MSCs. Rather, any method of isolating and culturing MSCs should be construed to be included in the present invention.

Antibiotics which can be added into the medium include, but are not limited to, penicillin and streptomycin. The concentration of penicillin in the culture medium, in a non-limiting embodiment, is from about 10 to about 200 units per ml. The concentration of streptomycin in the culture medium, in a non-limiting embodiment, is from about 10 to about 200 μg/ml.

In a non-limiting embodiment, as the mesenchymal stem cells are being cultured, the mesenchymal stem cells are contacted with an agent which stimulates, or activates, the mesenchymal stem cells to express increased amounts of an agent that inhibits, prevents, or destroys the growth of tumors. Such stimulating, or activating agents include but are not limited to, TNF-α, IFN-γ, or any other inflammatory agent. In a non-limiting embodiment, the stimulating, or activating agent, is TNF-α.

Agents that inhibit, prevent, or destroy the growth of tumors, which are expressed in increased amounts by the stimulated, or activated, mesenchymal stem cells, include, but are not limited to, TNF-α related apoptosis inducing ligand, or TRAIL, and Dickkopf-related protein-3, or DKK-3, Interleukin-24, or IL-24, CD82, and/or combinations thereof and/or biologically active fragments, derivatives, and analogues thereof. In a non-limiting embodiment, the agent that inhibits, prevents, or destroys the growth of tumors is TRAIL or a biologically active fragment, derivative, or analogue thereof. In another non-limiting embodiment, the agent that inhibits, prevents, or destroys the growth of tumors is DKK-3 or a biologically active fragment, derivative, or analogue thereof.

Although the scope of the present invention is not to be limited to any theoretical reasoning, Applicants have discovered that the stimulated, or activated mesenchymal stem cells express increased amounts of agents that inhibit, prevent, or destroy the growth of tumors, and that such stimulated, or activated, mesenchymal stem cells kill tumor cells with better efficacy than recombinantly produced agents, such as TRAIL, that inhibit, prevent, or destroy the growth of tumors. Furthermore, “cross-talk” between the stimulated or activated mesenchymal stem cells increased expression of TRAIL and DKK-3 further. For example, dead tumor cells triggered a “feed forward” increase in expression of TRAIL by the stimulated, or activated mesenchymal stem cells, over and above that provided by the initial contact of the mesenchymal stem cells with the stimulation or activation agent hereinabove described, such as TNF-α.

The stimulated, or activated mesenchymal stem cells are administered to the animal, such as, for example, a human, by any acceptable means of administration known to those skilled in the art. Such methods include, but are not limited to, direct administration of the stimulated or activated mesenchymal stem cells to the tumor, or by intravenous, intraperitoneal, intracardiac, intramuscular, intradermal, subcutaneous, or topical administration. In a non-limiting embodiment, when the stimulated or activated mesenchymal stem cells are used to treat bone cancer, the stimulated or activated mesenchymal stem cells may be administered directly to the bone affected by the cancer.

The stimulated, or activated mesenchymal stem cells are administered in an amount effective to inhibit, prevent, or destroy the growth of a tumor.

In a non-limiting embodiment, the stimulated, or activated mesenchymal stem cells are administered in an amount of from about 10³ to about 10¹⁰ cells.

The exact dosage of the mesenchymal stem cells is dependent on a variety of factors, including the age, weight, and sex of the patient, the type and location of the tumor being treated, and the extent and severity thereof.

The mesenchymal stem cells are administered in conjunction with an acceptable pharmaceutical carrier or excipient.

Suitable carriers and excipients include those that are compatible physiologically and biologically with the mesenchymal stem cells and with the patient, such as phosphate buffered saline and other suitable carriers or excipients. Other pharmaceutical carriers that may be employed, either alone or in combination, include, but are not limited to, sterile water, alcohol, fats, waxes, and inert solids. Pharmaceutically acceptable adjuvants (e.g., buffering agent, dispersing agents) also may be incorporated into a pharmaceutical composition including the mesenchymal stem cells. In general, compositions useful for parenteral administration are well known. (See, for example, Remington's Pharmaceutical Science, 17^{th} Ed., Mack Publishing Co., Easton, Pa., 1990). Alternatively, the mesenchymal stem cells may be introduced into a patient by implantable systems. (See, for example, Urquhart, et al., Ann. Rev. Pharmacol. Toxicol., Vol. 24, pg. 199 (1984).

In another non-limiting embodiment, the mesenchymal stem cells may be contained in a nanoparticle. Such nanoparticles may be formed by methods known to those skilled in the art, and administered by methods such as those hereinabove described.

Tumors which may be treated with the stimulated or activated mesenchymal stem cells include malignant (i.e., cancer) and non-malignant tumors.

In one non-limiting embodiment, the tumor is a non-malignant tumor.

In another non-limiting embodiment, the tumor is a malignant tumor, i.e., a cancerous tumor.

Cancers which may be treated with the stimulated, or activated, mesenchymal stem cells include, but are not limited to, breast cancer, including breast metastases, lung cancer, Kaposi's sarcoma, colorectal cancer, cervical cancer, B-cell malignancies, including multiple myeloma, glioma, melanoma, including melanoma metastases, hepatomas, prostate cancer, pancreatic cancer, kidney cancer, Ewing's sarcoma, and bone cancer, including osteosarcomas.

The stimulated, or activated, mesenchymal stem cells may be administered to a patient as a “one-time” therapy for the treatment of cancer. A one-time administration of the stimulated, or activated mesenchymal stem cells to the patient eliminates the need for chronic anti-tumor therapy.

In another non-limiting embodiment, multiple administrations of the mesenchymal stem cells are employed.

The invention described herein also encompasses a method of preventing or treating cancer by administering the stimulated or activated MSCs in a prophylactic or therapeutically effective amount for the prevention, treatment, or amelioration of cancer. An effective amount of MSCs can be determined by comparing the level of cancer in a recipient prior to the administration of MSCs thereto, with the level of cancer present in the recipient following the administration of MSCs thereto. A decrease, or the absence of an increase, in the level of cancer in the recipient with the administration of MSCs thereto, indicates that the number of MSCs administered is a therapeutic effective amount of MSCs.

In addition, Applicants also have discovered that, when the stimulated, or activated mesenchymal stem cells are administered in combination with a chemotherapeutic agent, there is provided a synergistic effect.

Thus, in accordance with another aspect of the present invention, there is provided a method of inhibiting, preventing, or destroying the growth of a tumor by administering the stimulated, or activated mesenchymal stem cells hereinabove described in conjunction with at least one chemotherapeutic agent. The mesencylmal stem cells and at least one chemotherapeutic agent are administered in amounts effective to inhibit, prevent, or destroy the growth of a tumor in the animal.

Chemotherapeutic agents which may be administered in combination with the stimulated, or activated mesenchymal stem cells include, but are not limited to, doxorubicin, cisplatin, mitoxantrone, mithramycin, daunorubicin, docetaxel, epirubicin, 5-fluorouacil (5-FU), VP16, the cyclo-oxygenase inhibitor Du-P697, idarubcin, irinotecan (CPT-11), cladribine, cytarabine, gemcitabine, thioguanine, thiotepa, fenretinide, lapatinib, sun tin b, oxaliplatin, paclitaxel, dacarbazine, angiogenesis inhibitors, such as anti-VEGF antibodies (e.g., Avastin, Lucentis), mapaturnumab, lexatumumab, agonistic antibodies which recognize TRAIL death receptors, lipoxygenase inhibitors such as MK886, agents which suppress HSP70, apigenin, baicalein, isoliquirit genin, kaempferol, quercetin, wogonin, cycloartenyl ferulate, silibinin, gossypol, cardamonin, zerumbone, nimbolide, halocynthiaxanthin, γ-tocotrienol (γ-T3), garcinol, combretastatin A-4, methyl jasmonate, docosahexaenoic acid (DHA), diosgenin, all-trans-retinyl acetate (RAc), 15-deoxydelta (12,14)-prostaglandin J2 (15dPGJ2), naphthoquinone epoxides, such as 2,3-epoxy-2,3-dihydrolpachol and 2,3-epoxy-2,3 dihydro-8-hydroxylapachol, maritinone, elliplinone, plumbagin, dibenzylideneacetone (DBA), mesalamine derivatives such as 2-methoxy-5-amino-N-hydroxybenzamide (or 2-14), dipyridamole, nutlin-3,5-aminoimidazole-4-carboxamide riboside (AICAR), rottlerin, and 17-allylamino-17-demethoxygeldanamycin (17-AAG). Examples of such chemotherapeutic agents are described further in Stolfi, et al.; Int. J. Mol. Sci., Vol. 13, pgs. 7886-7901 (Jun. 25, 2012); Zou, et al., Requl. Toxicol Pharmacol., PM1D No. 23000416 (E-pub. Sep. 18, 2012); Engesaeter, et al., PLOS One, Vol. 7, Issue 9, pgs. 1-12 (Sep. 20, 2012); and Kim, et al., Cancer Res., Vol. 72, No. 18, pgs. 4807-4817 (Sep. 15, 2012; E pub. Sep. 7, 2012); Qiu, et al., Int. J. Mol. Sci., Vol. 13, No. 7, pgs. 9142-9156 (2012-Epub. Jul. 20, 2012); Sung, et al., Exp. Cell Res., Vol. 318, No. 13, pgs. 1564-1576 (August 2012-Epub. Apr. 10, 2012); Whitson, et al., J. Nat. Prod., Vol. 75, No. 3, pgs. 394-399 (Mar. 23, 2012-Epub. Feb. 7, 2012); Hellwig, et al., Mol. Cancer Ther., Vol. 11, No. 1, pgs. 3-13 (January 2012); Taylor, et al., BMC Cancer, Vol. 11, pgs. 470-487 (Nov. 1, 2011); and Menke, et al., Cancer Res., Vol. 71, No, 5, pgs. 1883-1892 (Mar. 1, 2011), the contents of which are incorporated herein by reference.

In a non-limiting embodiment, the at least one chemotherapeutic agent is doxorubicin.

The stimulated, or activated mesenclymal stem cells may be administered in the amounts hereinabove described. The stimulated, or activated mesenchymal stem cells may be administered to the patient prior to the administration of the at least one chemotherapeutic agent, concurrently with the administration of the at least one chemotherapeutic agent, or subsequent to the administration of the at least one chemotherapeutic agent.

In a non-limiting embodiment, the mesenchymal stem cells and the at least one chemotherapeutic agent are administered separately, i.e., in separate pharmaceutical compositions.

The at least one chemotherapeutic agent is administered in an amount effective to inhibit, prevent, or destroy the growth of a tumor. The exact amount of chemotherapeutic agent is dependent upon a variety of factors, including the age, weight, and sex of the patient, the type of tumor being treated, and the extent and severity thereof.

The at least one chemotherapeutic agent may be administered in conjunction with an acceptable pharmaceutical carrier or adjuvant, such as those hereinabove described.

Tumors which may be treated by administering the stimulated, or activated mesenchymal stem cells and the at least one chemotherapeutic agent include the malignant and non-malignant tumors hereinabove described.

In another non-limiting embodiment, the stimulated, or activated mesenchymal stem cells may be administered in combination with agents, such as, but not limited to, the proteasome inhibitors MG132 and PS-341, that reduce the toxicity of or resistance to one or more of the agents (e.g., TRAIL, DKK-3, IL-24, and/or CD82) that are expressed in increased amounts by the stimulated, or activated mesenchymal stem cells, and/or reduce the toxicity of or resistance to one or more of the above-mentioned chemotherapeutic agents.

In another non-limiting embodiment, the stimulated, or activated mesenchymal stem cells may be administered in combination with one or more anti-cancer vaccines, including, but not limited to, cervical cancer vaccines, for example.

In yet another non-limiting embodiment, the stimulated, or activated mesenchymal stem cells may be administered in combination with polynucleotides (DNA or RNA), such as antisense polynucleotides and siRNA, for example, that inhibit DNA or RNA replication and/or transcription and/or translation in tumor cells, thereby further inhibiting, preventing, and destroying the growth of the tumor cells.

The invention now will be described with respect to the following drawings, wherein:

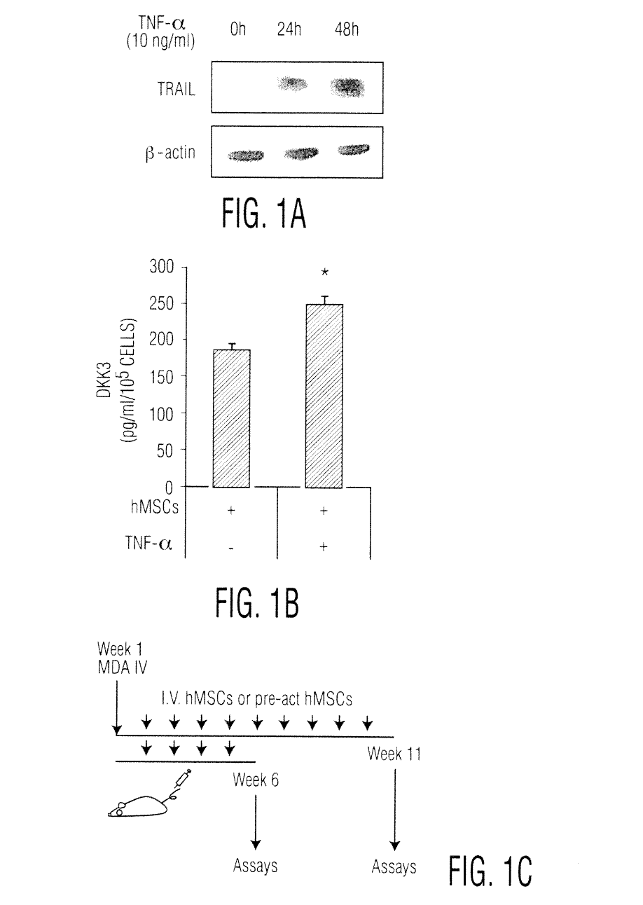

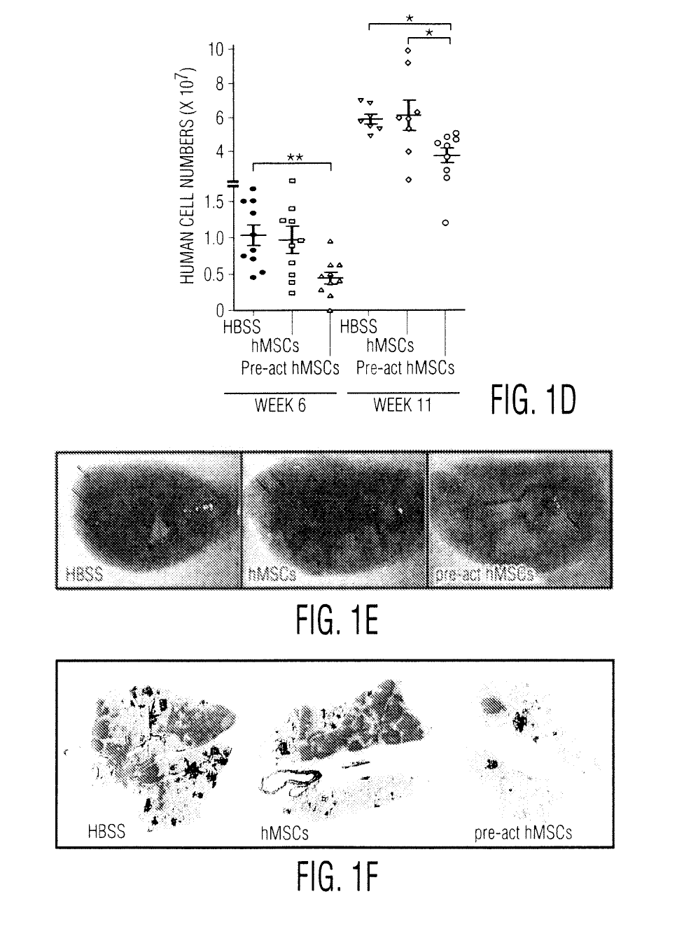

FIG. 1 IV Infusions of hMSCs Pre-activated with TNF-α Reduced Tumors in a Xenograft Mouse Model.

hMSCs were pre-activated by incubation with TNF-α (10 ng/mL) in 2% CM for 24 or 48 hrs. (B) ELISA assay for DKK3 in medium from hMSCs incubated as in (A) for 24 hrs. Values are means±S.D. (n=3; * p<0.05; two-tailed Student's t-test). (C) Schematic diagram. (D) Real-time PCR for human Alu sequences in mouse lungs. Values are means±S.D. for HBSS (n=10, week 6; n=7, week 10); for hMSCs (n=10, week 6; n=8, week 10) and for pre-act hMSCs (n=10, week 6; n=9, week 10); * p<0.05 and ** p<0.01; Kruskal-Wallis test). (E) Representative gross images of mouse lungs. The black arrows indicate tumor nodules. (F) Representative histology images (H&E staining) of lung sections.

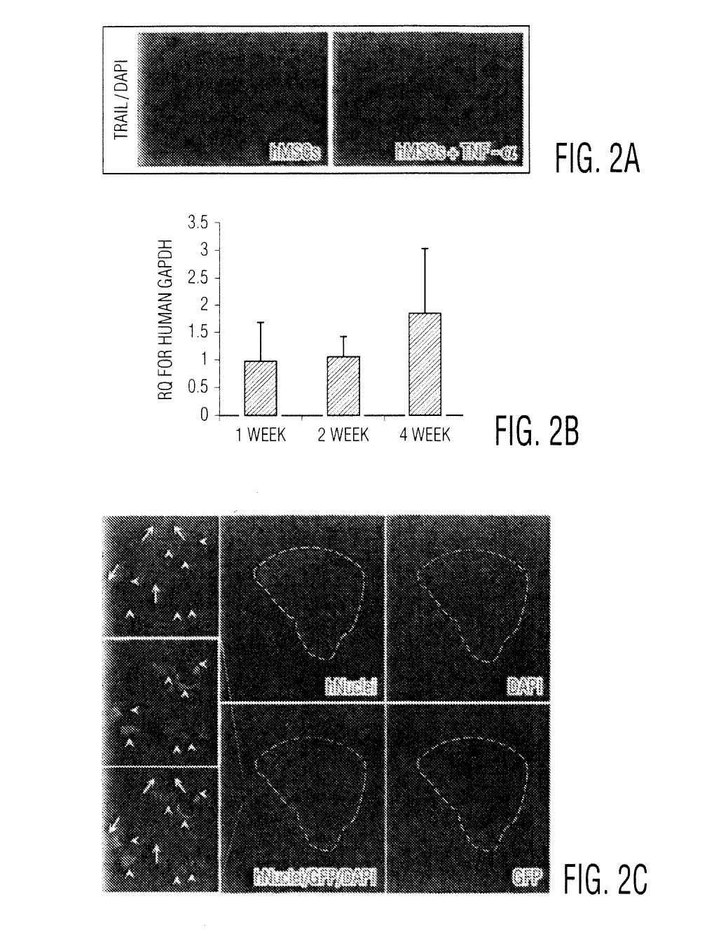

FIG. 2 (A) Representative immunofluorescent images of hMSCs following 24 hour activation with TNF-α. The cells were labeled for human TRAIL (red) and DAPI (blue). (B) Real time RT-PCR for human GAPDH expression in lungs following 2×10⁶ MDA i.v. infusion. Values are means±S.D. (n=3; one-way ANOVA). (C) Representative immunofluorescent images of tumor in a lung section. The section was labeled for human nuclei (red) and GFP (green). GFP expressing cells represent pre-activated hMSCs. The lungs were collected 24 hours after injection of pre-activated hMSCs.

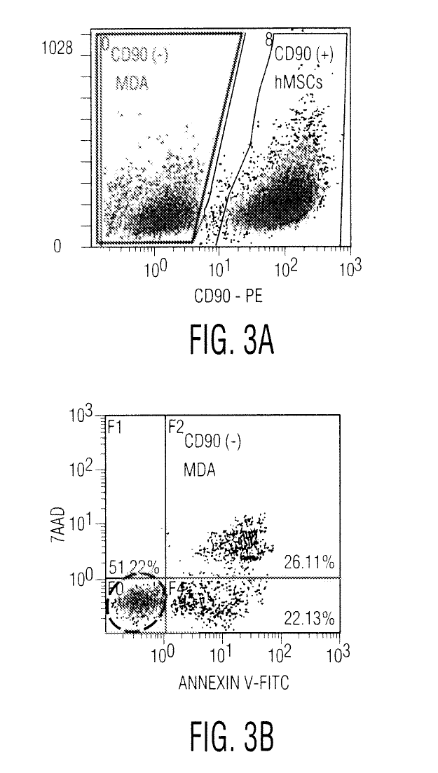

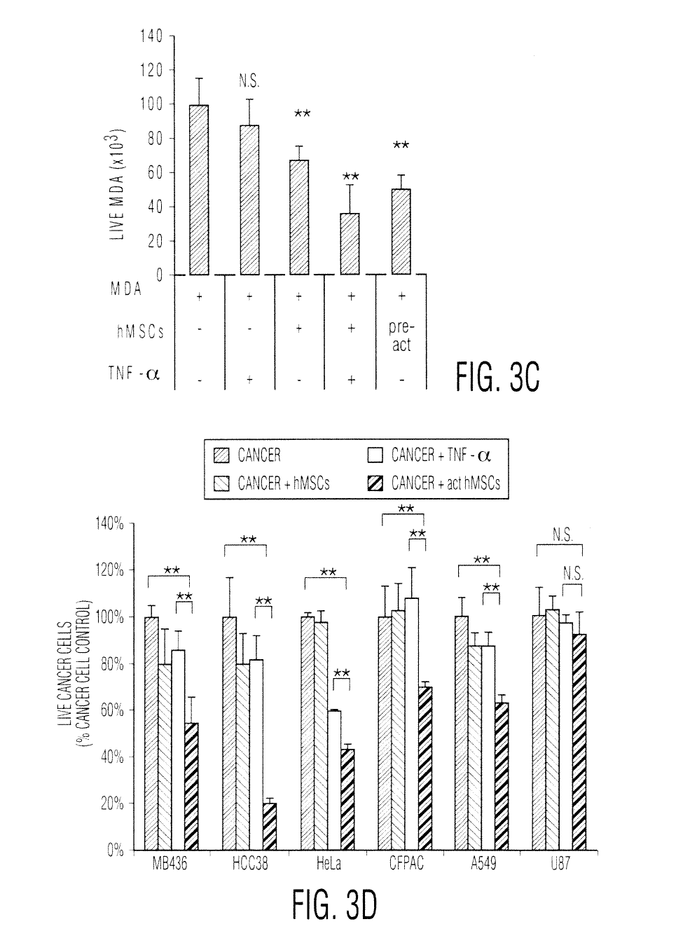

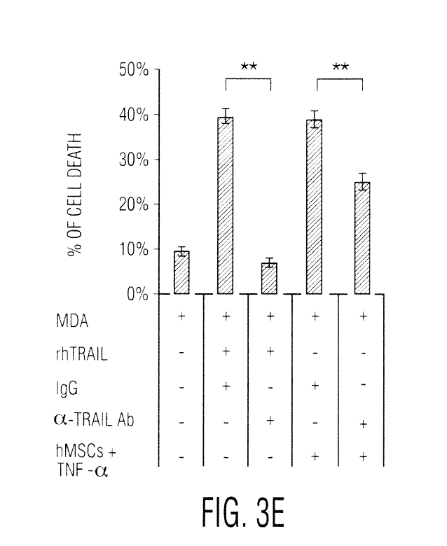

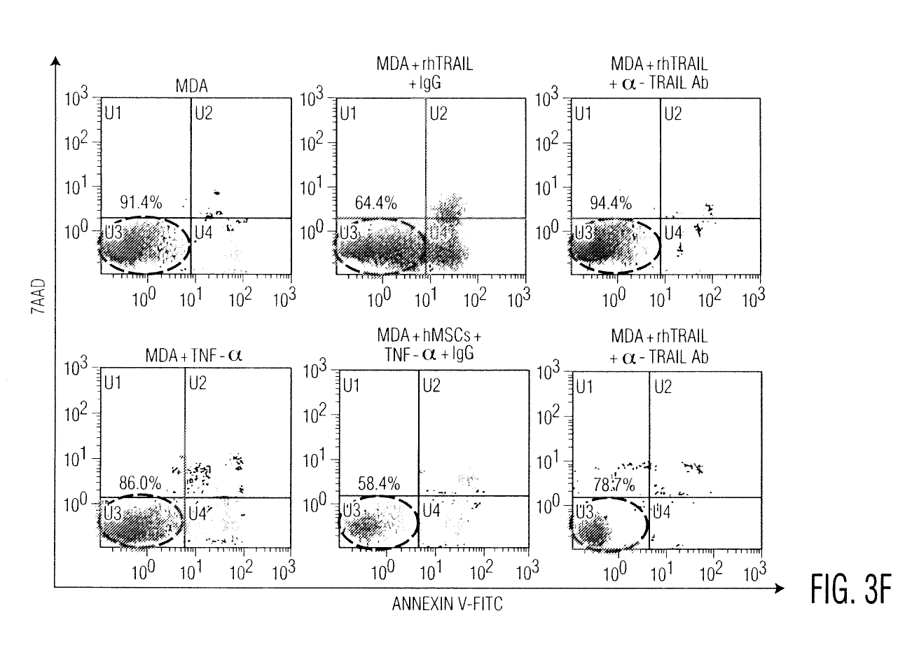

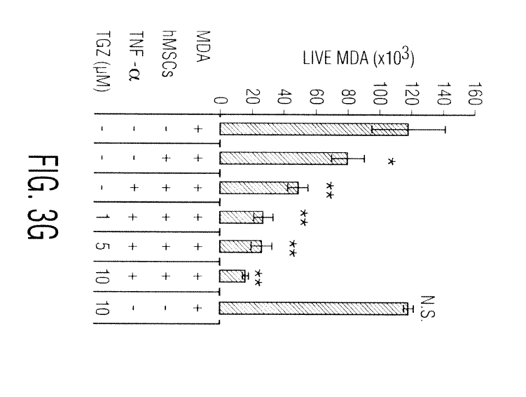

FIG. 3 hMSCs Induced TRAIL-Dependent Cell Death in MDA Cells.

(A) After co-culture of MDA (10⁵) with equal number of activated hMSCs for 24 his, cells were collected and labeled with anti-CD90 antibody to distinguish hMSCs to MDA. (B) Apoptosis was assayed by staining Annexin V-FITC & 7AAD in MDA cells identified as CD90 negative cells from FIG. 2A. (C) Number of live MDA cells from co-culture. Values are means±S.D. for cell counting (n=4; ** p<0.01; one-way ANOVA). (D) Live cancer cell numbers as percent of control following 24 hr co-culture with TNF-α, hMSCs or activated hMSCs. Values are means±S.D. for cell counting. (n=3 or 4; ** p<0.01; N.S.—Not Significant; one-way ANOVA). (E) Percentage of MDA cell death following 24 hr co-culture with pre-activated hMSCs and with IgG control antibody or anti-TRAIL antibody. Values are means±S.D. for cell counting (n=4; ** p<0.01; two-tailed Student's t-test). (F) Annexin V-FITC & 7AAD staining in MDA cells from experiment as in (E). (G) Number of live MDA cells following 24 hr co-culture with activated hMSCs and Troglitazone. Values are means±S.D. for cell counting (n=4; p<0.05; ** p<0.01; one-way ANOVA).

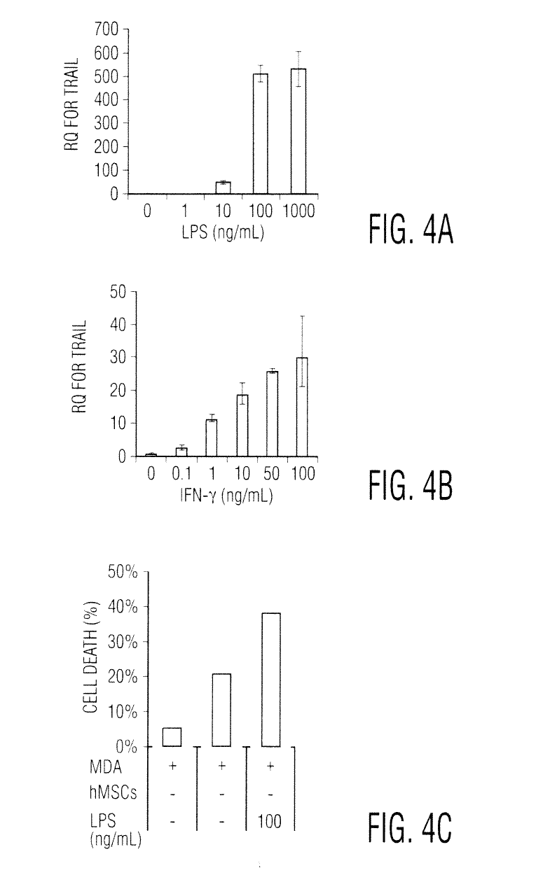

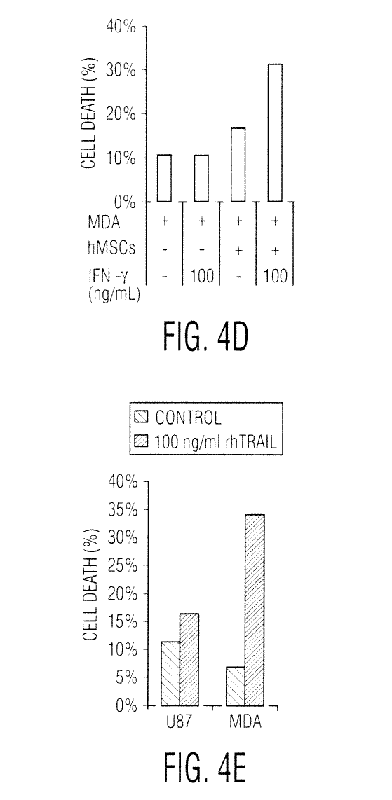

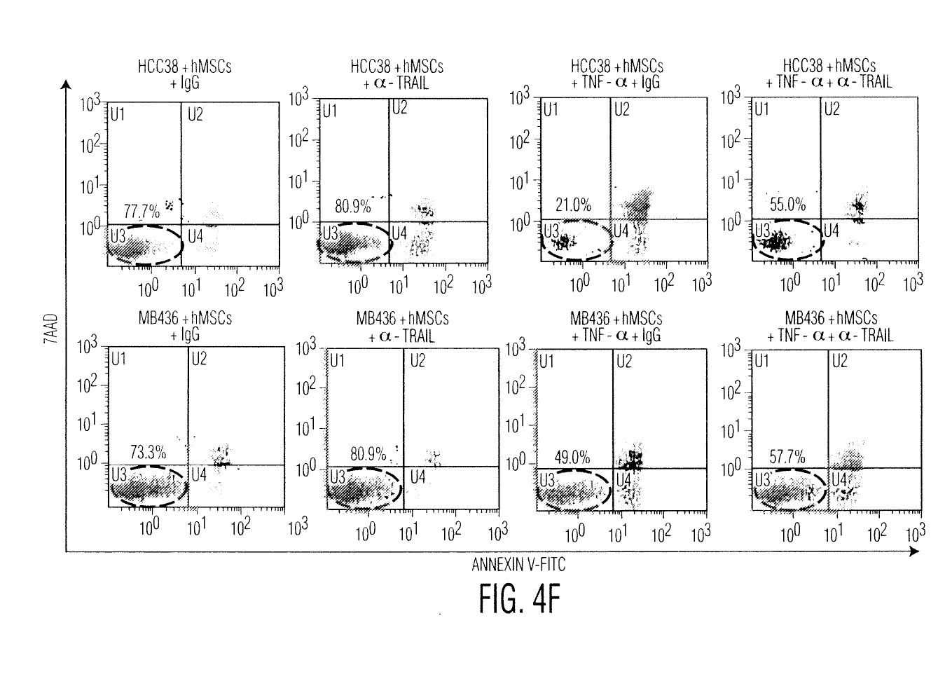

FIG. 4 (A-B) Real time RT-PCR for TRAIL in hMSCs following 24 hour incubation with LPS (A) or IFN-γ (B) Values are means±S.D. for triplicates of the assay. (C-D) Percentage of MDA cell death following 24 hour co-culture with hMSCs activated with 100 ng/mL of LPS (C) or 100 ng/mL of IFN-γ (D). (E) Percentage of U97 and MDA cell death following 24 hour co-culture with 100 ng/ml rhTRAIL. (F) Annexin V-FITC & 7AAD staining in HCC38 and MDA-MB-436 (MB436) cells following 24 hr co-culture with hMSC or activated hMSCs and with IgG control antibody or anti-TRAIL antibody.

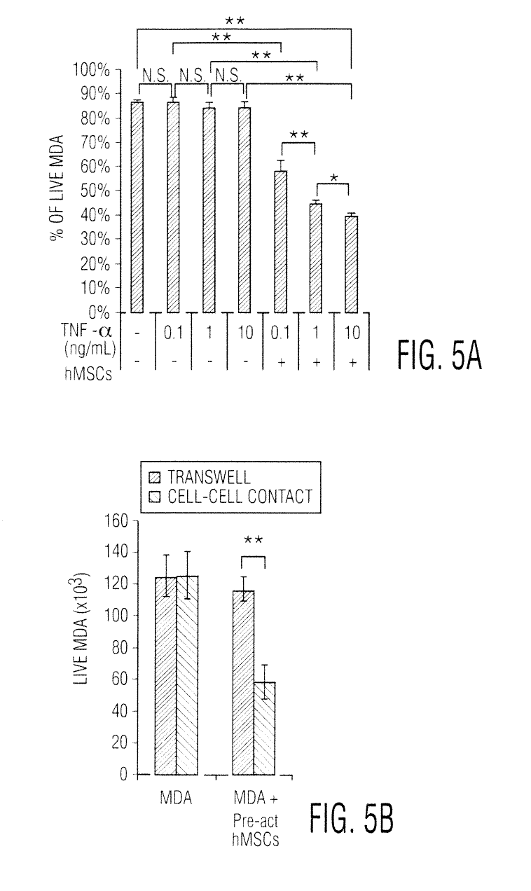

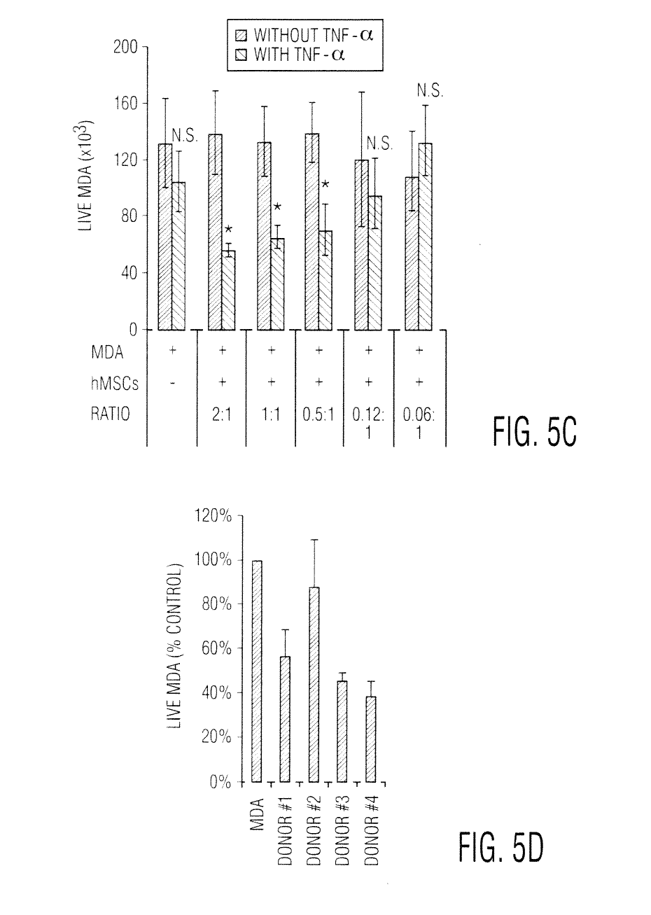

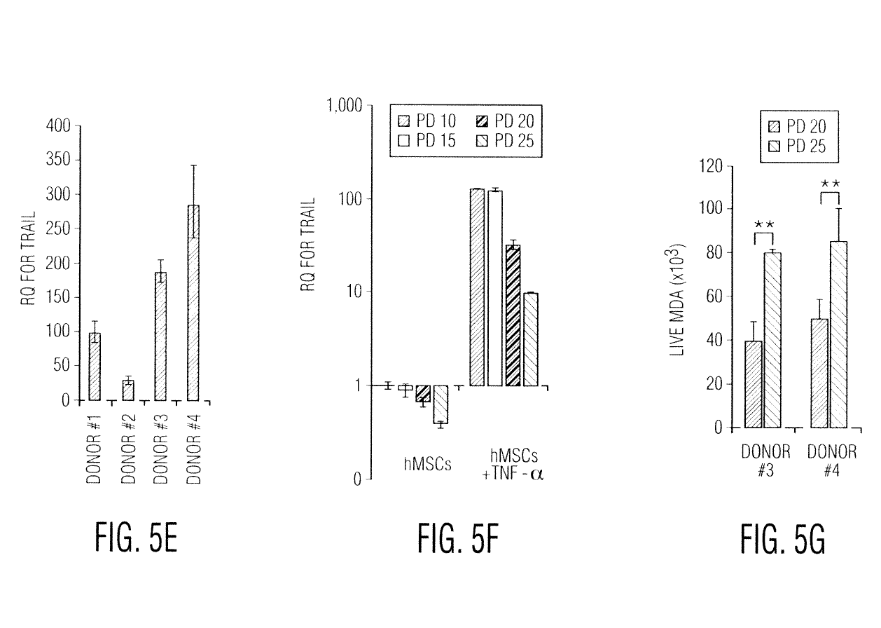

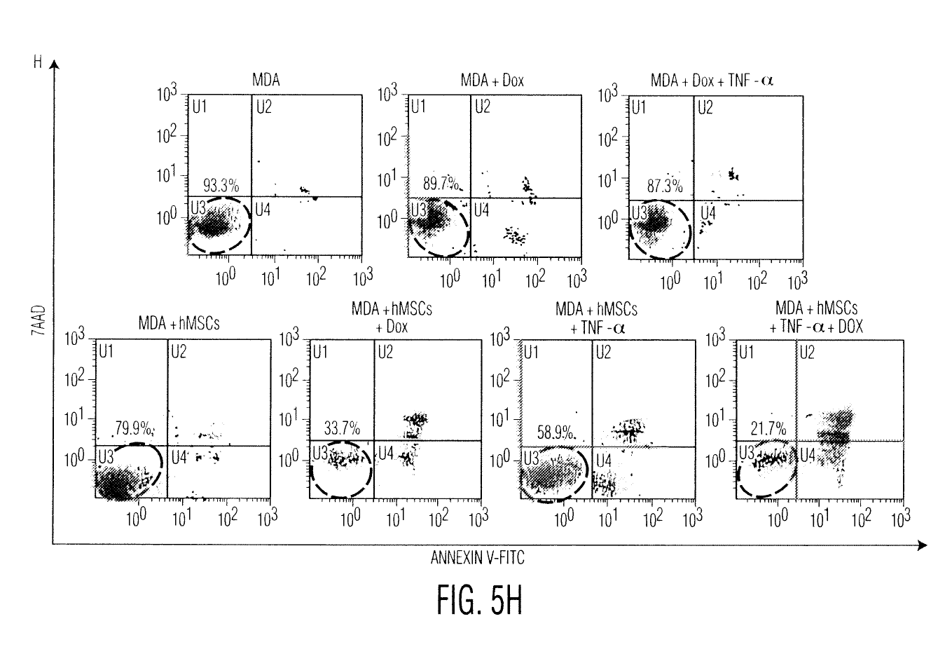

FIG. 5 Conditions for Co-Culture with MDA and hMSCs and Variations among hMSC Preparations

Cells were cultured for 24 or 48 hrs as in FIG. 2. (A) Effect of TNF-α concentration on live MDA cells recovered from 48 hr co-cultures. Values are means±S.D. for cell counting (n=4;* p<0.05 and ** p<0.01; one-way ANOVA). (B.) Lack of effect in transwell co-cultures for 24 hrs. Values are means±S.D. for cell counting (n=4; ** p<0.01; two-tailed Student's test). (C) Decrease in live MDA cells recovered from co-cultures with increasing ratio of hMSCs to MDA. Values are means±S.D. for cell counting (n=4; * p<0.05; N.S.—Not Significant; one-way ANOVA). (D) Variations in recovery of live MDA cells from co-cultures with pre-act MSCs from different donors. Values are means±S.D. (n=3). (E) Variations in TRAIL expression is pre-act hMSCs from different donors. Values are means±S.D. for triplicates of the assay. (F) Decrease in TRAIL expression in pre-act hMSCs after expansion in culture through 25 population doublings (PD). Values are means±S.D. for triplicates of the assay. (G) Decrease in effectiveness in co-cultures of pre-act hMSCs expanded through 25 population doublings. Values are means±S.D. for cell counting (n=4; ** p<0.01; two-tailed Student's t-test). (H) Decrease in recovery of live MDA cells from co-cultures after pre-incubation of the MDA cells for 24 hrs with doxorubicin (100 ng/mL). (I) Percentage of live MDA cell growth from experiment as in (H). Values are expressed as means±S.D. (n=4; ** p<0.01; N.S.—Not Significant; one-way ANOVA).

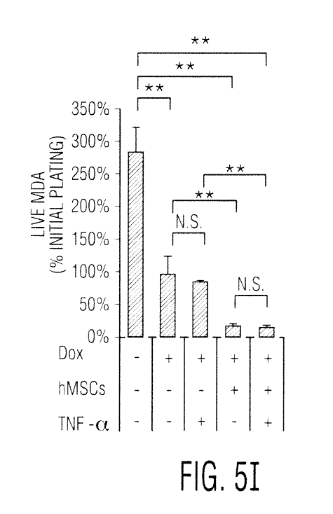

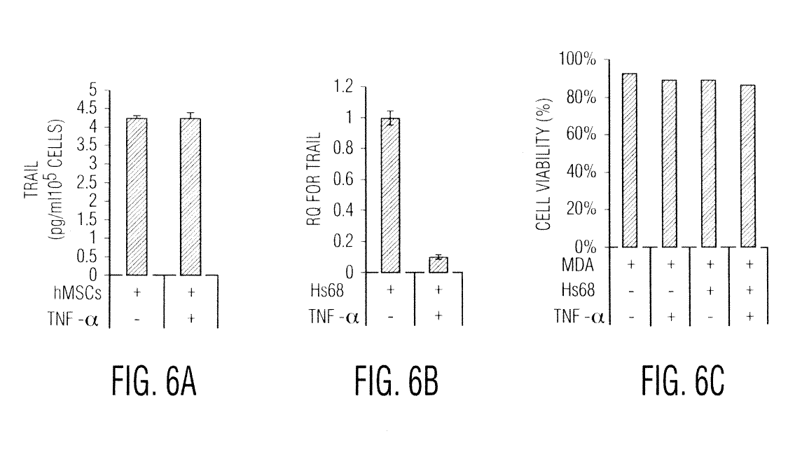

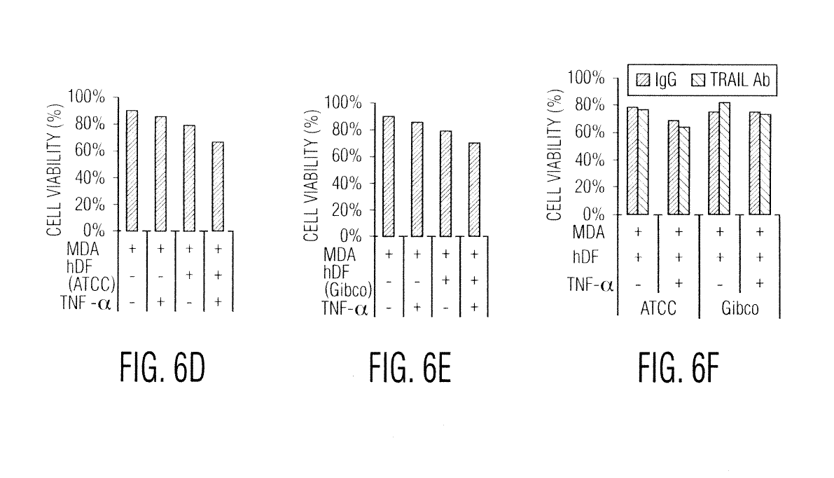

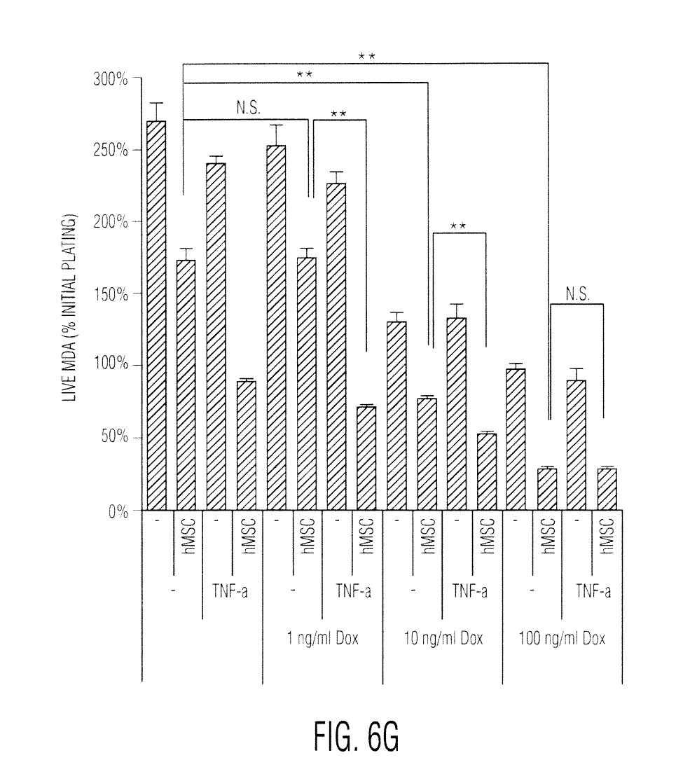

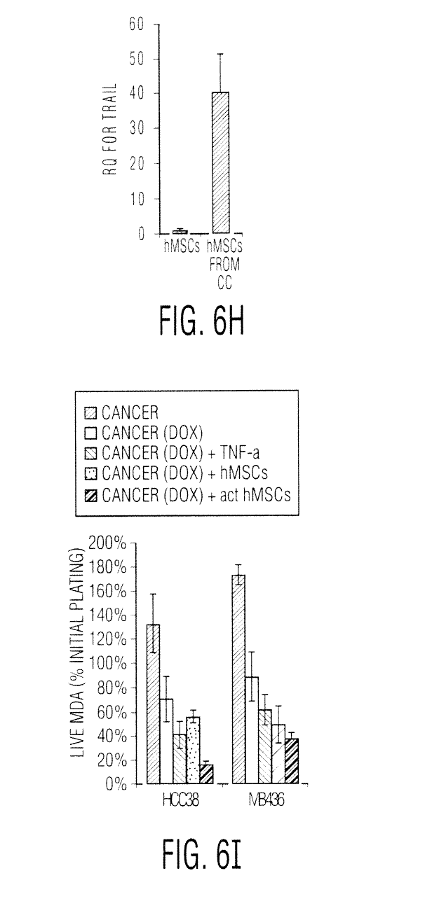

FIG. 6 (A) ELISA assay for TRAIL in conditioned medium of hMSCs or hMSCs treated with 10 ng/mL TNF-α. Values are means±S.D.; n=3. (B) Real time RT-PCR for TRAIL in human fibroblasts (Hs68) following 24 hour incubation with TNF-α (10 ng/mL). Values are means±S.D. for triplicates of the assay. (C) Cell death assay in MDA cells following 24 hour co-culture with Hs68 fibroblasts by staining with Annexin V-FITC and 7AAD. (D) Cell death assay in MDA cells following 24 hour co-culture with human dermal fibroblasts (hDF; ATCC) by staining with Annexin V-FITC and 7AAD. (E) Cell death assay in MDA cells following 24 hour co-culture with human dermal fibroblasts (hDF; Gibco) by staining with Annexin V-FITC and 7AAD. (F) Cell death assay in MDA cells following 24 hour co-culture with two different hDFs with either IgG or TRAIL neutralizing antibody, by staining with Annexin V-FITC and 7AAD. (G) Live MDA cells following 24 hour with or without doxorubicin pre-treatment followed by co-culture with hMSCs with or without TNF-α. Values are means±S.D. for cell counting (n=4; ** p<0.01; N.S.—Not Significant; one-way ANOVA). (H) Real-time RT-PCR for TRAIL expression in control hMSCs and hMSCs following 24 hr co-culture (CC) with MDA without TNF-α. Values are means±S.D. for triplicates of the assay. (I) Percentage of live MDA cell growth following 24 hrs cultures with hMSCs or act hMSCs after pre-incubation of the HCC38 and MDA-MB-436 (MB436) cells for 24 hrs with doxorubicin (100 ng/mL). Values are means±S.D. for cell counting (n=4).

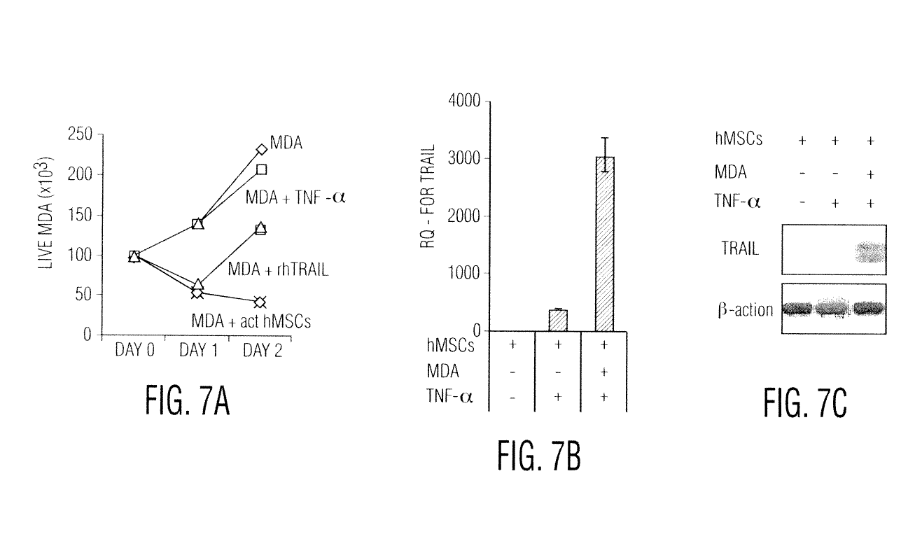

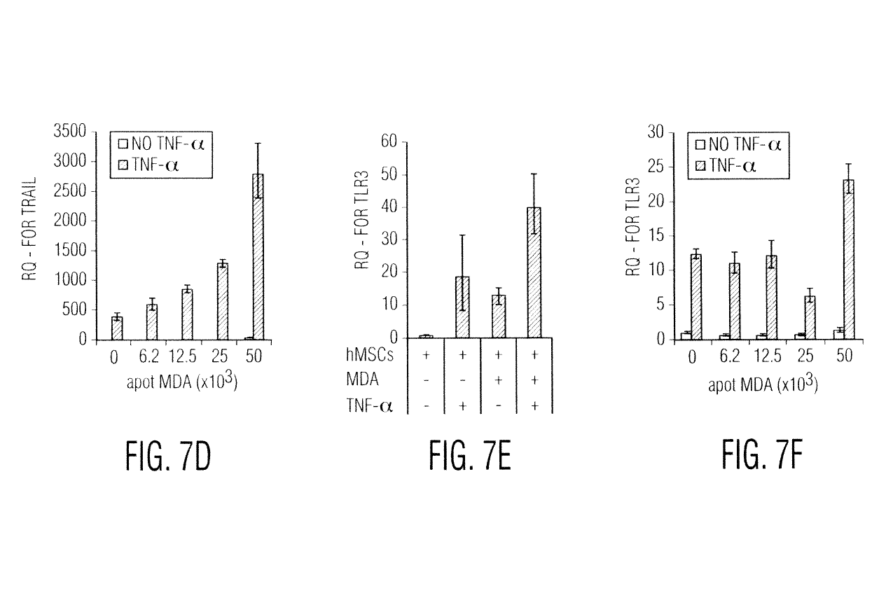

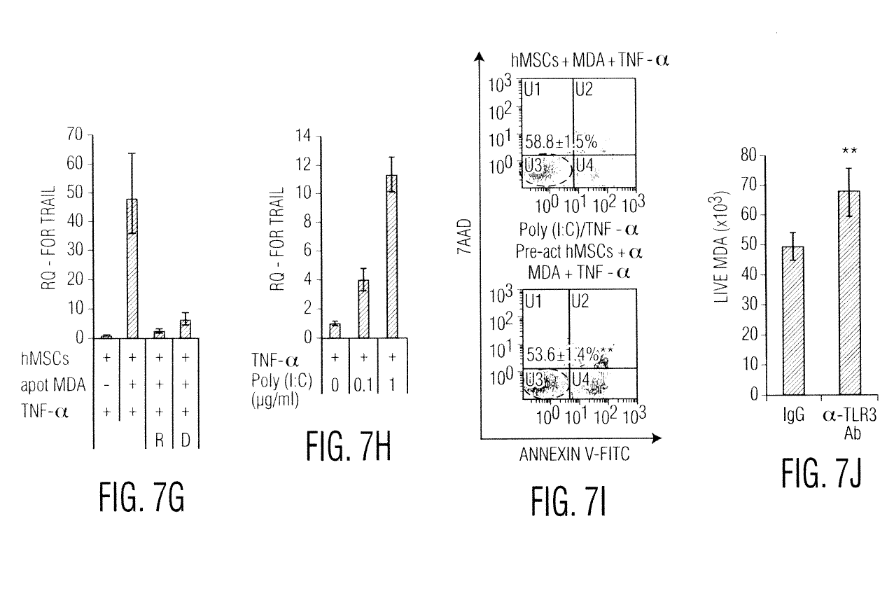

FIG. 7 Expression of TRAIL on hMSCs Increased upon Co-Culture with MDA.

(A) Live MDA cells following 48 hr co-culture with activated hMSCs or rhTRAIL (200 ng/mL). (B) Real-time RT-PCR for TRAIL expression in hMSCs following 24 hr treatment of TNF-α or co-culture with MDA and TNF-α. Values are means±S.D. for triplicates of the assay. (C) Western analysis for TRAIL in hMSCs from experiment in (B). (D) Real-time RT-PCR for TRAIL in hMSCs following 24 hr incubation with apoptotic MDA cells (apot MDA) and with or without TNF-α. Values are means±S.D. for triplicates of the assay. (E) Real-time RT-PCR for TLR-3 in hMSCs from experiment as in (B). Values are means±S.D. for triplicates of the assay, (F) Real-time RT-PCR for TLR3 in hMSCs from experiment as in (D). Values are means±S.D. for triplicates of the assay. (G) Real-time RT-PCR for TRAIL in hMSCs following 24 hr incubation with apot MDA or apot MDA pre-treated with RNase (R) or DNase (D). Values are means±S.D. for triplicates of the assay. (H) Real-time RT-PCR for TRAIL in hMSCs following 24 hr treatment with poly (I:C). Values are means±S.D. for triplicates of the assay. (I) Cell death assay in MDA by labeling with Annexin V & 7AAD following 24 hr co-culture with poly (I:C)/TNF-α pre-activated hMSCs and TNF-α. Values are means±S.D. (n=3; ** p<0.01; two-tailed Student's t-test). (J) Number of live MDA cells following 24 hr co-culture with activated hMSCs and mouse IgG or TLR3 blocking antibody (5 μg/mL). Values are means±S.D. (n=3; ** p<0.01; two-tailed Student's t-test).

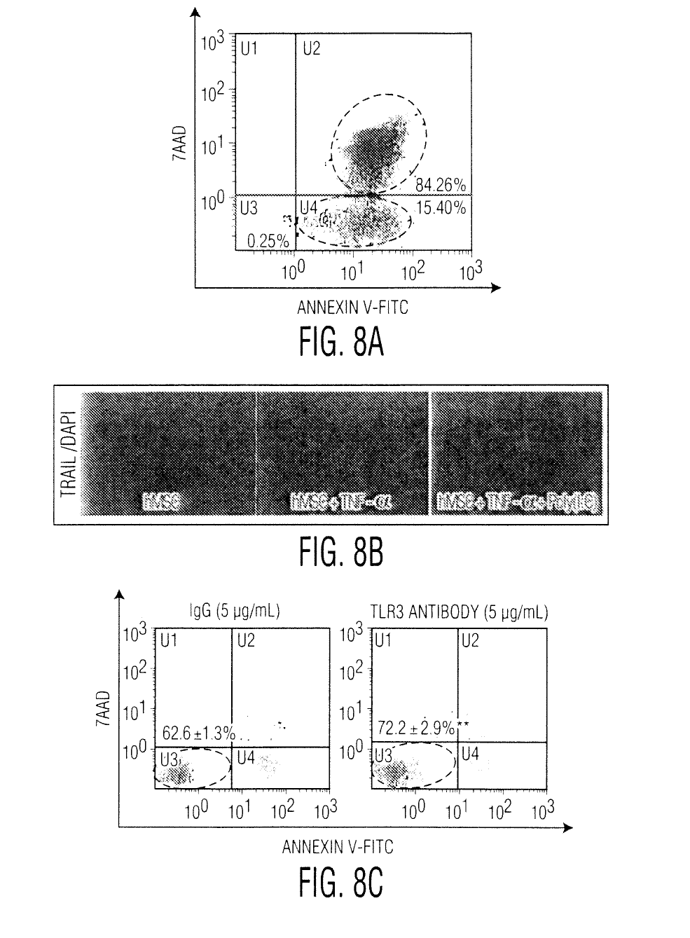

FIG. 8 Cell death assay in MDA by staining with Annexin V-FITC and 7AAD following 24 hour treatment with 100 ng/mL rhTRAIL in serum free α-MEM. (B) Representative immunofluorescent images of hMSCs following 24 hour activation with TNF-α or a combination of TNF-α and Poly (I:C). The cells were labeled for human TRAIL (red) and DAPI (blue). (C) Cell death assay in MDA by labeling with Annexin V and 7AAD following 24 hour co-culture with activated hMSCs and a mouse IgG (5 μg/mL) or TLR3 blocking antibody (5 μg/mL). Values are mean±S.D. (n=3; ** p<0.01; two-tailed student t-test).

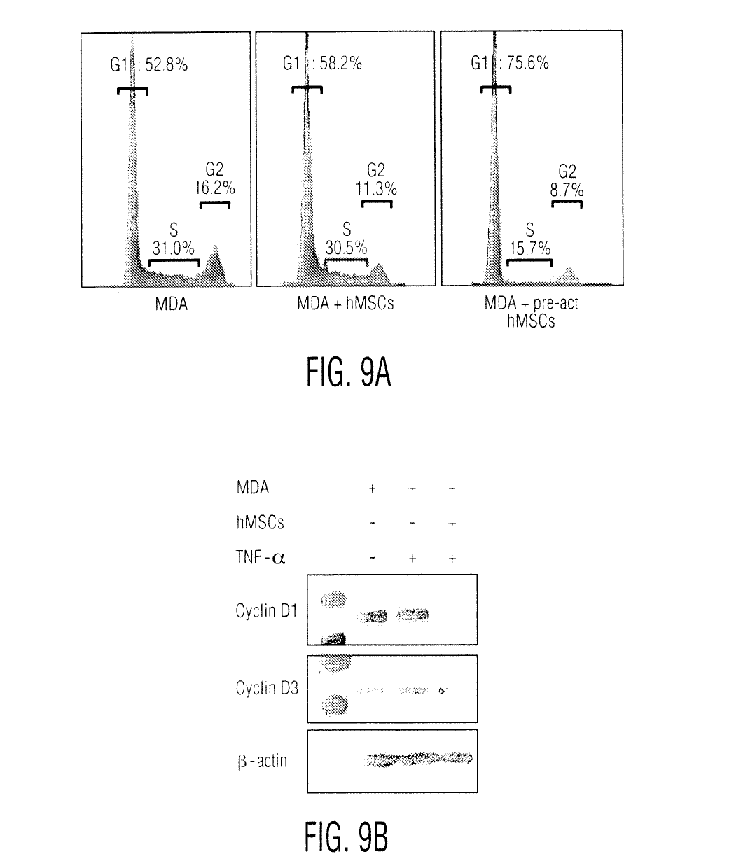

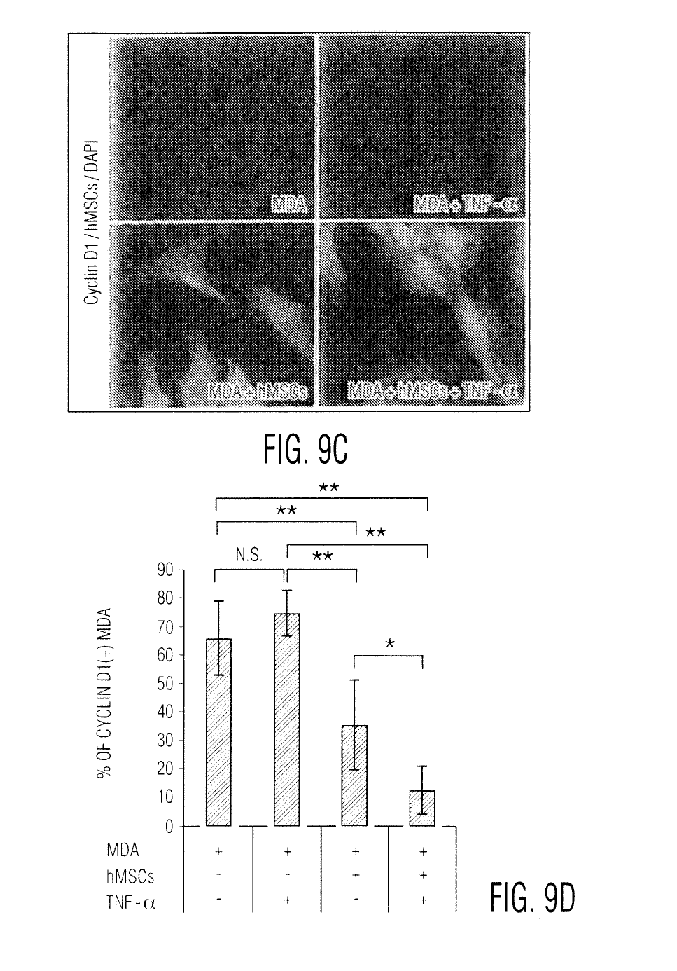

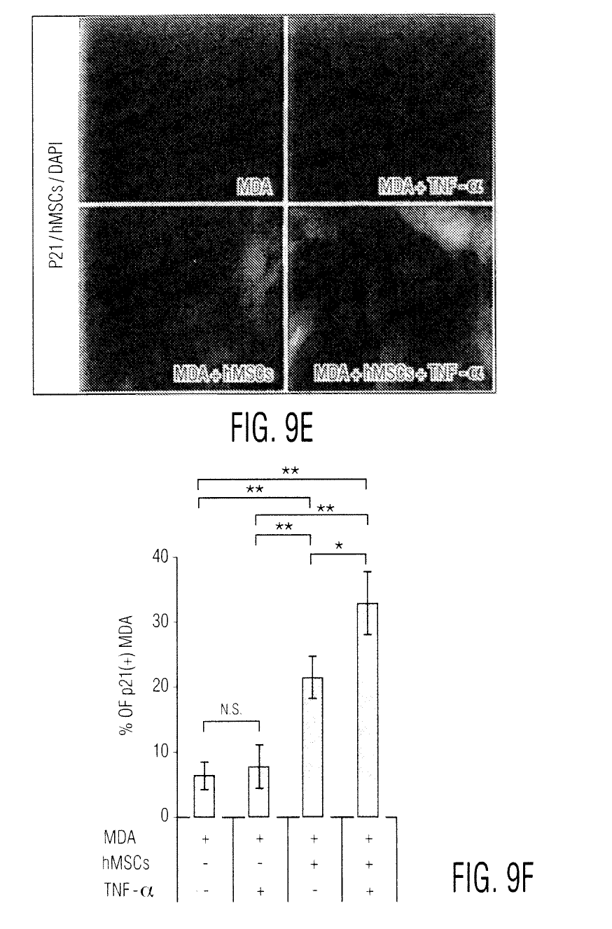

FIG. 9 hMSCs Activated with TNF-α Inhibited Cell Cycle Progression in MDA Cells

(A) Cell cycle was assayed in adherent viable MDA cells following 24 hr co-culture with hMSCs or pre-act hMSCs. (B) Western blot analyses for cyclin D1 and cyclin D3 levels in MDA cells following 24 hr incubation with TNF-α or co-culture with pre-act hMSCs. (C-F) MDA cells were cultured with TNF-α or co-culture with GFP-labeled activated hMSCs for 24 hrs. (C) IF staining for cyclin D1 (Red) and GFP (green). (D) Quantification of data of cyclin D1 expressing MDA cells from experiment in (C). Values are means±S.D. for three random fields with at least 50 cells per field scored for each sample (n=3; * p<0.05; ** p<0.01; N.S.—Not significant; one-way ANOVA). (E) IF staining for p21 (Red) and GFP (green) from experiment in (C). (F) Quantification of data of p21 expression in MDA cells from experiment in (E). Values are means±S.D. for three random fields with at least 50 cells per field scored for each sample (n=3; * p<0.05; ** p<0.01; N.S.—Not significant; one-way ANOVA).

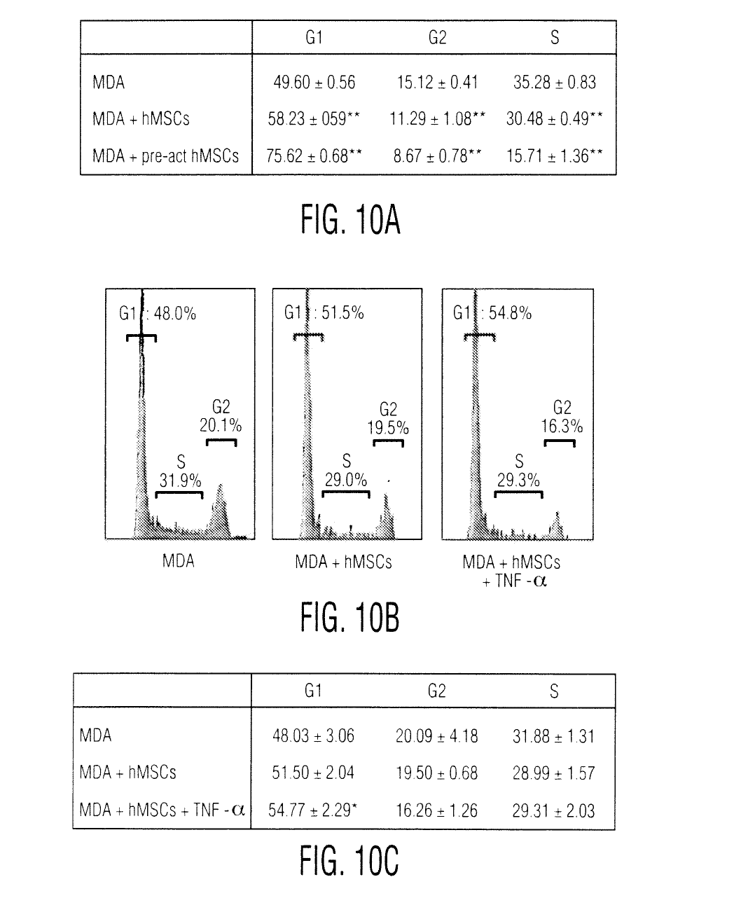

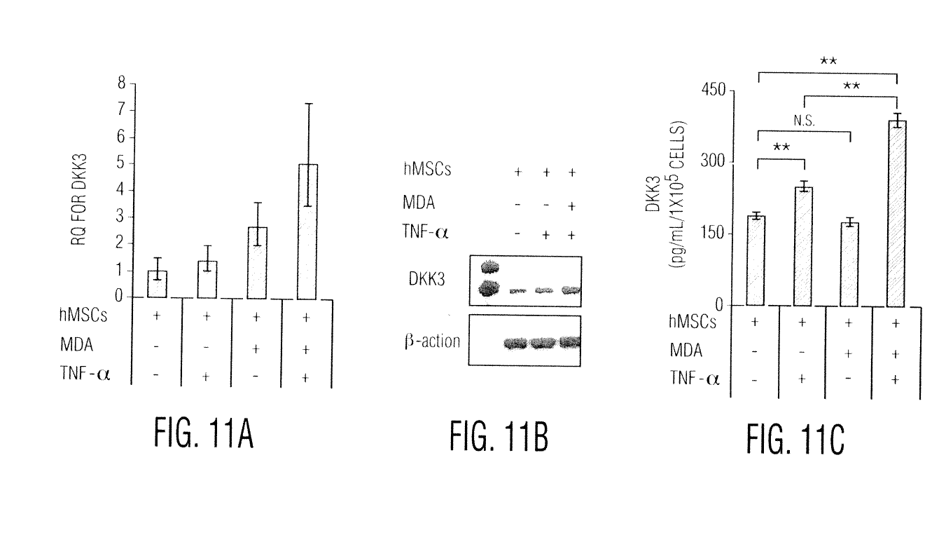

FIG. 10 (A) Cell cycle was assayed in MDA cells following 24 hours co-culture with TNF-α, hMSCs or activated hMSCs. Values are means±S.D. (n=3; ** p<0.01: one-way ANOVA). (B and C) Cell cycle was assayed in MDA cells following 24 hours co-culture with TNF-α, hMSCs or activated hMSCs in transwells. Values are means±S.D. (n=3; * p<0.05; one-way ANOVA).

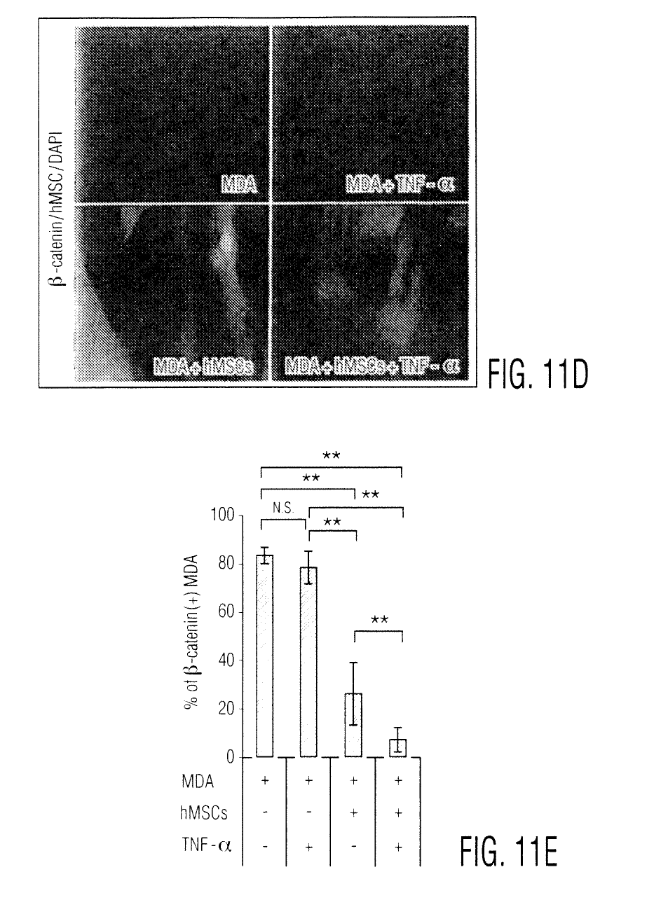

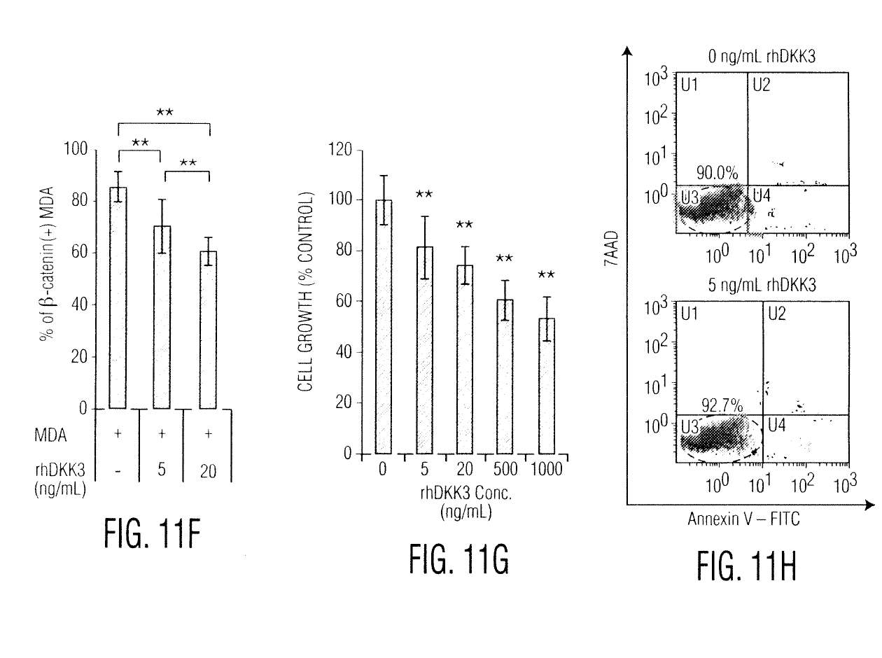

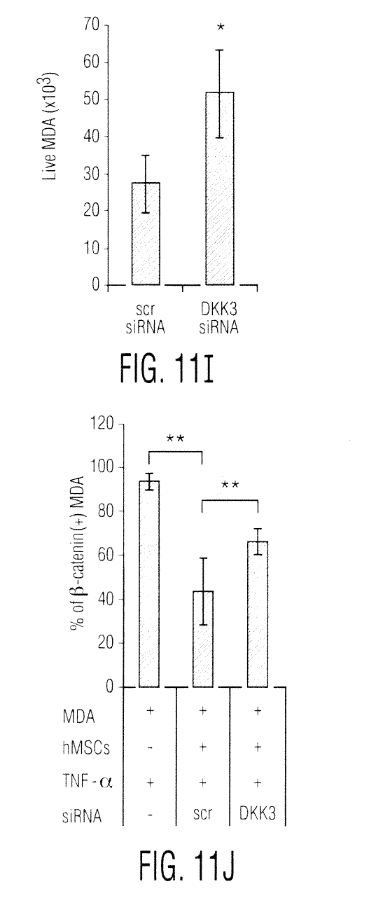

FIG. 11 DKK-3 Expressed by Activated hMSCs in Co-Cultures Decreased Proliferation on MDA Cells.

(A-D) MDA cells were cultured with TNF-α or co-culture with GFP-labeled activated hMSCs for 24 hrs. (A) Real-time RT-PCR for DKK3. Values are means±S.D. for triplicates of the assay. (B) Western blots for DKK3. (C) ELISA assay for DKK3 in medium. Values are means±S.D. (n=3; ** p<0.01; N.S.—Not significant; one-way ANOVA). (D) IF staining for β-catenin (Red) and GFP (green). (E) Quantification of data of β-catenin expressing MDA cells from experiment in (D). Values are means±S.D. for three random fields with at least 50 cells per field scored for each sample (n=3; ** p<0.01; N.S.—Not Significant; one-way ANOVA). (F) Quantification of β-catenin expressing MDA cells following 24 hr incubation with rhDKK3 (R&D Systems) in 2% CM. Values are means±S.D. for five random fields with at least 50 cells per field scored for each sample (n=5; ** p<0.01; one-way ANOVA). (G) MTT assay showing the rate of cell proliferation of MDA cells following 24 hr incubation with rhDKK3. Values are means±S.D. (n=9; ** p<0.01; one-way ANOVA). (H) Cell death assay in MDA cells following 24 hr treatment with rhDKK3 (5 ng/mL) by labeling with Annexin V & 7AAD. (I) Number of live MDA cells following 24 hr co-culture with activated hMSCs transduced with scr siRNA (control) or DKK3 siRNA. Values are means±S.D. (n=3; * p<0.05; two-tailed Student's t-test). (J) Quantification of β-catenin expressing MDA cells from the experiment as in (I). Values are means±S.D. for five random fields with at least 50 cells per field scored for each sample (n=5; ** p<0.01; one-way ANOVA).

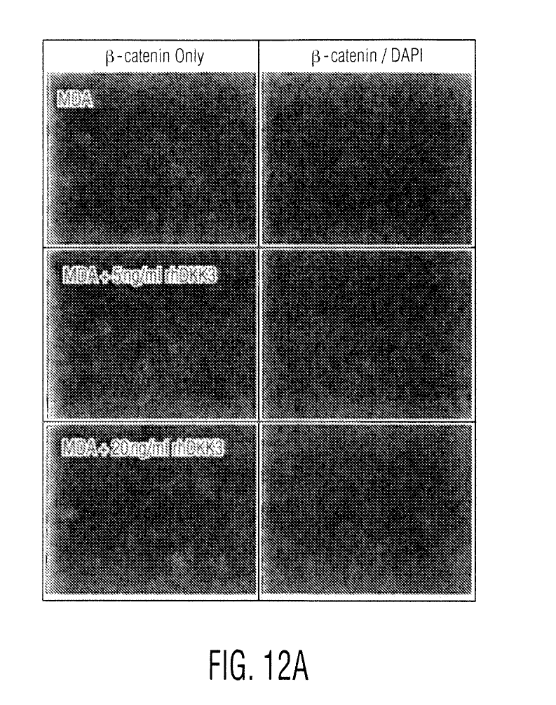

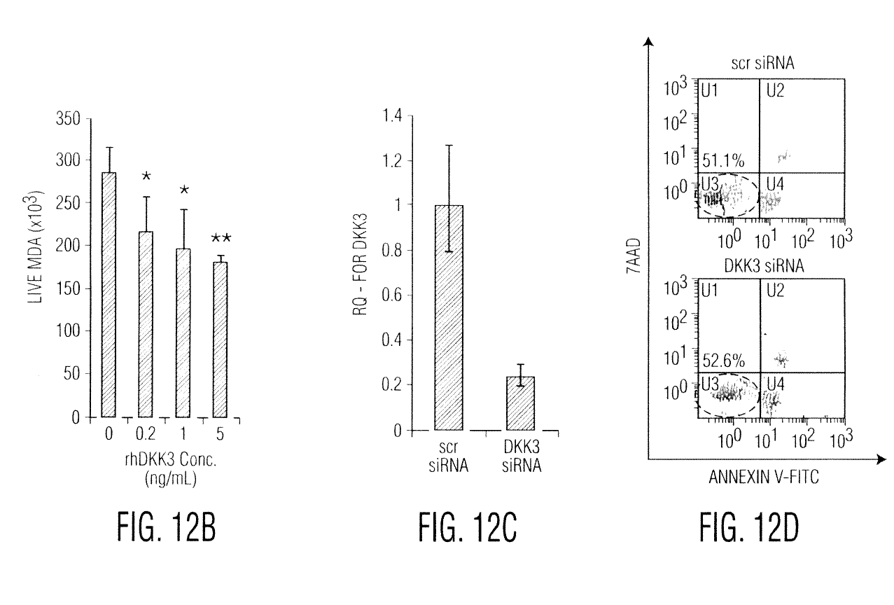

FIG. 12 (A) Representative immunofluorescent images for β-catenin (Red) in MDA cells treated with different concentrations of rhDKK3. (B) Number of live MDA cells following incubation with different concentrations of rhDKK3. Values are means±S.D. for cell counting (n=4; * p<0.05; ** p<0.01; one-way ANOVA). (C) Real time RT-PCR for DKK3 in hMSCs following scr siRNA (control) or DKK3 siRNA transduction. Values are means±S.D. for triplicates of the assay. (D) Labeling with Annexin V and 7AAD in MDA cells following co-culture with activated hMSCs transduced with either scr siRNA or DKK3 siRNA.

The invention now will be described with respect to the following example; however, it is to be understood that the scope of the present invention is not intended to be limited thereby.

### EXAMPLE

### Experimental Procedures

### Cell Preparations

hMSCs were prepared as previously described (Lee et al., Cell Stem Cell, Vol. 5, pgs 54-63 (2009)). MDA-MB-231, MDA-MB-436, Hela, HCC38, A549, CFPAC, U87, Hs68 and two different human dermal fibroblasts were purchased from ATCC (Manassa, Va.) and Gibco (Grand Island, N.Y.).

### Animals

Six week old male NOD/SCID mice (NOD.CB17-Prkdcscid/J) from the Jackson Laboratory (Bar Harbor, Me.) were used under a protocol approved by the Institutional Animal Care and Use Committee of Texas A&M Health Science Center College of Medicine.

Lung Xenograft Model, hMSC Infusion and Tissue Collection

Animals were injected through a tail vein with 2×10⁶ MDA cells in order to initiate lung metastases. Beginning one week later, mice were injected weekly intravenously with HBSS, 2×10⁶ hMSCs, or pre-activated hMSCs for a total of 4 or 9 injections, a protocol similar to that used previously (Loebinger et al., Cancer Res., Vol. 69, pgs. 4134-4142 (2009). Pre-activated hMSCs were prepared by incubating the cells for 24 his before injection with recombinant human TNF-α protein (TNF-α; 10 ng/mL; R&D Systems; Minneapolis, Minn.) in 2% culture medium (2% CM; alpha-MEM containing 2% fetal bovine serum (FBS; lot-selected for rapid growth of MSCs; Atlanta Biologicals, Inc, Norcross, Ga.), 100 units/mL penicillin, 100 μg/mL streptomycin, and 2 mM L-glutamine (Invitrogen, Grand Island, N.Y.)]. For injection, hMSCs were harvested with 0.25% trypsin/1 mM EDTA and re-suspended at 2×10⁶ cells in 200 μL of HBSS. One week after the last injection, lungs were collected for assay.

### Lung Tissue Preparations for Human Alu Assay and Immunofluorescence Staining

One week after the last injection of hMSCs or HBSS, mice were euthanized with a lethal dose of ketamine/xylazine (200/20 mg/kg final; 0.06 mL) and lungs were collected in tissue extraction buffer (10 mM Tris HCl, pH 8.0; 0.1 M EDTA, pH 8.0; 0.5% SDS; 20 μg/mL RNaseA; and 10 mg/mL proteinase K) for DNA isolation. Genomic DNA was extracted from lungs and used for real time PCR assay for human Alu detection, as previously described.

Lung samples were homogenized (PowerGen Model 125 Homogenizer; Fisher Scientific) in tissue extraction buffer (10 mM Tris HCl, pH 8.0; 0.1 M EDTA, pH 8.0; 0.5% SDS; 20 μg/mL RNaseA; and 10 mg/mL proteinase K) and incubated at 50° C. overnight with shaking (200 rpm). Genomic DNA (gDNA) was extracted by mixing 0.5 mL of homogenized sample with 0.5 mL phenol/chloroform solution (pH 6.7, Invitrogen) and centrifugation at 15,300×g for 5 minutes in 2 mL phase-lock gel tubes (Phase Lock Gel; 5 Prime, Inc; Gaithersburg, Md.). gDNA suspended in the supernatant was precipitated with half volume of 2.5 M ammonium acetate and equal volume of 100% ethanol overnight at 4° C. The precipitates were washed with ice-cold 75% ethanol and re-suspended in sterile H₂O.

For immunofluorescence staining, upon euthanization with ketamine/xylazine, mice were perfused through the left ventricle with 20 mL PBS and then through the right ventricle with 5 mL PBS. A catheter with 20 G needle was inserted into the trachea and the lungs were filled with approximately 5 mL (until lung was fully inflated) 30% Optimal Cutting Temperature (OCT) Compound (Sakura Finetek U.S.A.; Torrance, Calif.) in PBS. Fully expanded lungs were sealed with a suture, embedded in OCT Compound, and frozen using an isopentane bath placed on dry ice. Frozen tissue blocks were stored in −80° C.

Real Time PCR Assays with gDNA for Human Alu

gDNA extracted from mouse lungs was used for real-time PCR assays for human Alu (hAlu) sequences (Lee et al., 2009). The assay contained 10 μL Taqman Universal PCR Master Mix (Applied Biosystems; Carlsbad, Calif.), 900 nM each of the forward and reverse primers, 250 nM TaqMan probe and 200 ng of gDNA.

For internal controls, human and mouse specific GAPDH sequences were assayed using 10 μL of SYBR Green Master Mix (Applied Biosystems), 200 nM each of the forward and reverse primers and 200 ng of gDNA. Reactions were incubated at 50° C. for 2 minutes and at 95° C. for 10 minutes followed by 40 cycles at 95° C. for 15 seconds and 60° C. for 1 minute. Real-time amplification was analyzed on 7900HT fast real-time PCR system (Applied Biosystems). Standard curves were generated by adding serial dilutions of MDA into fresh mouse lung samples without any cancer cells prior to homogenization (Lee et al., 2009).

Sequences for primers and probes were previously described (Lee et al., 2009).

Real-Time RT-PCR Analysis for Selected mRNAs

Approximately 200 ng of total RNA extracted from cultured cells was used to synthesize double-stranded complementary DNA (cDNA) by reverse transcription (SuperScript III; Invitrogen). cDNA was analyzed by real-time PCR using TaqMan Universal PCR Master Mix (Applied Biosystems). For the assays, reactions were incubated at 50° C. for 2 minutes and at 95° C. for 10 minutes followed by 40 cycles at 95° C. for 15 seconds and 60° C. for 1 minute. For relative quantitation of gene expression, human-specific GAPDH primers and probe (Taq-Man Gene Expression Assays ID, Hs00266705_g1) were used. The other PCR primers and probes were human DKK3 (Hs00247426_m1), human TRAIL (TNFSF10: Hs00921974_m1) and human TLR3 (Hs01551078_m1) all purchased from the same source (Applied Biosystems).

### Western Blot Analysis

Cultured cells were sorted using FACS after CD90-PE staining. Sorted samples were lysed (Cell Extraction Buffer; Invitrogen) and sonicated for 15-20 seconds (Ultrasonic Processor, 130W; Cole-Parmer; Vernon Hills, Ill.). Approximately 10 μg of protein sample was mixed with tracking dye (Blue Loading Buffer pack; Cell Signaling Technology, Inc.) as suggested by the manufacturer. The samples were heated at 97° C. for 5 minutes. Denatured protein samples were separated by electrophoresis on polyacrylamide gels (NuPAGE® Bis-Tris Gels; Invitrogen) and proteins transferred to Invitrolon™ PVDF membrane (Pore size—0.45 μm; Invitrogen) using Novex® NuPAGE® SDS-PAGE Gel System (Invitrogen). Membranes were then blocked for 1 hour at room temperature (RT) using blocking buffer that contained 5% non-fat dry milk (Cell Signaling Technologies, Inc.) in Tris-buffered saline with 0.1% Tween-20 (TBST). After blocking, membranes were incubated overnight at 4° C. with primary antibodies that were diluted in 5% BSA in TBST. The membranes were washed with TBST three times for 5 minutes each, and incubated with secondary antibodies, which were diluted in blocking buffer, for 1 hour at RT. After secondary antibody incubation, the membranes were washed with TBST three times and developed using West-Q Chemiluminescent Substrate Kit (Gendepot, Inc.; Barker, Tex.) for 1 minute. Images were taken using Molecular Imager VersaDoc™ MP 4000 (Bio-rad Laboratories). The membranes were washed with TBST three times and stripped using Restore™ Western Blot Stripping buffer (Thermo Fisher Scientific, Inc.) for 15 minutes at 37° C., followed by three more washes with TBST. The membranes were blocked for 1 hour using blocking buffer and incubated with β-actin primary antibody conjugated with HRP (1:10,000) diluted in blocking buffer followed by imaging as described above. The primary antibodies used in this study were anti-human TRAIL rabbit monoclonal antibody (C9289), anti-human cyclin D1 mouse monoclonal antibody (DCS6), anti-human cyclin D3 mouse monoclonal antibody (DCS22), all purchased from Cell Signaling Technologies. The anti-human DKK-3 biotinylated goat IgG was purchased from R&D Systems. The antibodies were used in concentrations that were recommended by manufacturers. For secondary antibodies, HRP-linked anti-rabbit IgG, anti-mouse IgG and anti-biotin IgG (Cell Signaling Technologies; 1:2,000) were used.

### ELISA Assays

Conditioned media from cultured MDA and hMSCs were collected after 24 hour incubation. For the DKK-3 assay, culture media were filtered (Spin-X® Centrifuge Tube Filter, 0.22 μm; Corning, Inc) and the protein level was detected by ELISA (Human Dkk-3 DuoSet; R&D Systems). For detection of TRAIL, the filtered medium was concentrated to 4 times (Vivaspin 6 (5 kDa MWCO; GE Healthcare; UK) and assayed by ELISA (Human TRAIL Quantikine ELISA kit; R&D Systems).

### Immunofluorescence Staining

Frozen lungs embedded in OCT Compound were cut in 10 μm sections (Microm HM 560; Thermo Fisher Scientific) and every 10^{th} section was collected onto slides (POLYSINE® Microscope slides; Thermo Fisher Scientific). The collected sections were stored in −80° C. For immunohistofluorescence, the slides were dried briefly, washed with 1M phosphate buffered saline (PBS) for 5 minutes and fixed with cold 4% paraformaldehyde (PFA) for 10 minutes at RT. The slides were washed with PBS three times for 5 minutes each and treated with 3% H₂O₂ solution in PBS for 20 minutes at RT. Following H₂O₂ treatment, the slides were washed with PBS three times then blocked with blocking solution (5% goat serum with 0.4% Triton-X in PBS) for 1 hour at RT. The slides were incubated with primary antibodies diluted in blocking solution overnight at 4° C. The slides were then washed with PBS three times followed by secondary antibody incubation diluted in blocking solution for 1 hour at RT. After secondary antibody incubation, the slides were washed with PBS and mounted using VECTASHIELD® Mounting Medium with DAPI (Vector Laboratories, Inc.; Burlingame, Calif.).

For immunofluorescence staining, hMSCs were cultured in CCM overnight in 150 mm diameter plates and replated at 10,000 cells/cm² in chambered slides (Lab Tek II Chamber Slide™ System; Nunc Thermo Scientific, Rochester, N.Y.). After incubating hMSCs for 2 to 24 hours in CCM, an equal number of MDA cells was added to hMSC-containing chambers in α-MEM with 2% FBS, 100 units/mL penicillin, 100 μg/mL streptomycin, and 2 mM L-glutamine with or without TNF-α (10 ng/mL) for 24 hours. After co-culture, the cells were washed with PBS twice and fixed with either methanol for anti-TRAIL antibody or 4% PFA for all other antibodies. The slides were further processed for immuno-staining as described above. The antibodies used for immunochemistry were anti-human nuclei mouse monoclonal antibody (Millipore, Billerica, Mass.) anti-GFP-Alexa Fluor® rat monoclonal antibody (RQ2; MBL Co. Ltd, Woburn, Mass.), anti-human TRAIL rabbit polyclonal antibodies (C92B9; Cell Signaling) anti-human cyclin D1 mouse monoclonal antibody (DCS6; Cell Signaling), anti-human p21 mouse monoclonal antibody (DCS60; Cell Signaling) and anti-human β-catenin mouse monoclonal antibodies (BD Bioscience, Sparks, Md.). Alexa Fluor® antibodies (Invitrogen) were used for secondary antibodies.

### Microscope Imaging

Immunostained sections and cells were imaged using Olympus FluoView 300 confocal microscope (Japan).

### Co-Cultures of Human MSCs and MDA Cells

hMSCs were plated at 10⁵ cells in CCM in 6-well plates and incubated for 4 hrs. For pre-activation, hMSCs were incubated at 37° C. for 24 hrs in 2% CM containing 10 ng/mL TNF-α. For co-culture, an equal number of MDA cells in 2% CM with or without TNF-α (10 ng/mL) were added to hMSC containing wells. For transwell cultures, hMSCs were plated at 10⁵ cells in the upper compartment (Transwell, 0.4 μm pore size; Corning; Corning, N.Y.), while an equal number of MDA cells was plated in the lower compartment in 2% CM with or without TNF-α (10 ng/mL). After 24 hrs, supernatants and cells were collected. For some experiments, MDA cells were treated with 1 to 100 ng/mL doxorubicin (Sigma-Aldrich) in 2% CM for 24 his at 37° C., prior to co-culture with hMSCs. In other experiments, hMSCs were treated with either apoptotic MDA cells or 1 μg/mL Polyinosinic-Polycytidylic acid [Poly(I:C); Sigma-Aldrich; St. Louis, Mo.] with or without TNF-α (10 ng/ml) for 24 hrs in 2% CM.

### Preparation of Apoptotic MDA Cells

MDA cells were plated in serum free α-MEM with 100 ng/mL rhTRAIL (R&D Systems). After 24 hrs at 37° C., floating cells were collected and washed by centrifugation at 500×g for 5 minutes. The pellet was re-suspended in 2% CM and plated on hMSC containing wells. For RNase and DNase treatment, apoptotic MDA cells were washed by centrifugation, re-suspended in 0.2 mL PBS containing either 20 μg of RNase (QIAGEN, Valencia, Calif.) or 30 units of DNase (QIAGEN), and incubated for 2 hrs at 37° C. The cells were washed by centrifugation and re-suspended in 2% CM before adding to hMSC containing wells.

### Flow Cytometry

hMSCs and MDA cells from co-culture experiments were suspended in PBS and incubated with CD90-PE (Clone Thy1/310; Beckman Coulter) for 45 minutes on ice, washed with PBS by centrifugation, incubated at RT for 20 minutes with 300 ng/mL annexin V (AnnexinV-FITC Apoptosis Detection Kit; Sigma-Aldrich) and 4 μg/mL 7-aminoactinomycin D (7AAD; Sigma-Aldrich.), and analyzed by flow cytometry (Cytomics FC500; Beckman Coulter).

RNA Extraction from Cultured Cells and Real Time RT-PCR Analysis

hMSCs and MDA cell were separated by cell sorting (FACS, MoFlo™ XDP; Beckman Coulter; Brea, Calif.) after labeling with CD90-PE for 45 minutes on ice. RNA was extracted using RNeasy Mini Kit (QIAGEN). See Supplemental Information for real time RT-PCR analysis.

### Cell Cycle Analysis

Both hMSCs and MDA cells were co-cultured for 24 hrs at 37° C. Conditioned media and apoptotic cells were aspirated and adherent cells were lifted using 0.25% trypsin/1 mM EDTA followed by re-suspension in ice-cold 75% ethanol for fixation for 1 hr on ice. The fixed cells were washed with PBS followed by centrifugation, and incubated overnight at 4° C. with 10 μg/mL propidium iodide (P.I.; Sigma-Aldrich) before flow cytometry.

Transfections with siRNA

hMSCs (5×10⁴ cells/well in 6 well plate) were transfected by incubating 6 hrs with 20 nM siRNA for DKK3 Trilencer-27 (Origene Technologies, Inc., Rockville, Md.) or Universal Scrambled Negative Control siRNA Duplex (Origene) using Lipofectamine RNAiMax reagent (Invitrogen).

### Antibody Blocking Assay

hMSCs (5×10⁴ cells/well in 6 well plate) were treated with either TLR3 antibody (5 μg/mL; eBioscience, Inc; San Diego, Calif.), TRAIL antibody (25 μg/mL; BD Biosciences, Inc; San Jose, Calif.), or normal mouse IgG (5 or 25 μg/mL; PeproTech, Inc; Rocky Hill, N.J.) for 30 minutes at 37° C. in 2% CM with or without TNF-α (10 ng/mL) prior to co-culture with an equal number of MDA cells for 24 hrs.

Results

Intravenous Infusions of hMSCs Pre-Activated with TNF-α Reduced the Size of Tumors in a Xenograft Mouse Model

First, expression of TRAIL and DKK3 protein was up-regulated in hMSCs after incubating the cells with 10 ng/mL TNF-α (FIGS. 1A, 2A and 1B). To explore whether hMSCs pre-activated with TNF-α (pre-activated hMSCs) have the ability to induce cell death in cancer cells, a xenograft model of human breast cancer metastasis with progressive tumor growth (FIG. 2B) was induced by injecting MDA cells (2×10⁶) intravenously into NOD/SCID mice (FIG. 10). The model was shown previously to respond to hMSCs transduced virally to express TRAIL (Loebinger et al., Cancer Res., Vol. 69, pgs. 4134-4142 (2009). A week after MDA injection, either male control hMSCs (2×10⁶) or male pre-activated hMSCs (2×10⁶) were injected intravenously weekly for 4 or 9 consecutive weeks. Both pre-activated hMSCs and control hMSCs were found by immunofluorescence (IF) staining in the tumors 1 day after IV injection (FIG. 2C). Some of the cells migrated to tumor sites and incorporated into the tumors (FIG. 2C); however, the cells did not persist. After 1 week, less than 0.01% of the infused cells were detected by qPCR for the human Y chromosome in the injected male hMSCs (not shown). Therefore, qPCR was employed for repetitive human Alu sequences to provide quantitative estimates of the growth of the human female breast cancer cells in the mouse lung. The distribution of cancer cells and hMSCs in other organs was not examined. The results demonstrated that pre-activated hMSCs suppressed tumor cell growth compared to HBSS control group at both early and late time points (FIG. 10). Controls of hMSCs that were not exposed to TNF-α and that did not express TRAIL (FIGS. 1A and 2A) did not have any statistically significant effect on tumor burden compared to the HBSS control group (FIG. 1D). The gross pictures and histology images (H&E staining) of lungs demonstrated that injection of pre-activated hMSCs decreased the number of tumor nodules (FIGS. 1E and 1F). The decrease in tumor burden seemed larger by gross images and histology of the lung (FIGS. 1E and 1F) than by the assays for Alu sequence (FIG. 1D), perhaps because the assays for Alu sequences underestimated the tumor burden in the control samples as a result of necrosis and DNA degradation at the center of large tumors. Therefore, the results suggested that the hMSCs suppressed the tumors by homing to the tumor site.

hMSCs Induced TRAIL-Dependent Apoptosis in MDA Cells

To determine whether hMSCs can induce apoptosis in MDA cells in vitro, MDA cells were directly co-cultured with hMSCs, hMSCs in the presence of TNF-α (activated hMSCs) or pre-activated hMSCs. After 24 hour (hr) co-culture, hMSCs and MDA cells were distinguished by antibodies to CD90, an epitope expressed by hMSCs but not by MDA cells (FIG. 3A), and apoptosis of MDA cells was analyzed using 7AAD and Annexin V staining (FIG. 3B). When MDA cells were cultured with activated hMSCs or pre-activated hMSCs, the apoptosis increased remarkably with a corresponding decrease in the number of live MDA cells (FIG. 3C). Naïve hMSCs co-cultured with MDA cells also reduced the number of live MDA cells, but to a lesser extent than activated hMSCs (FIG. 3C). In addition, TRAIL expression in hMSCs was induced by incubation of the cells with two other pro-inflammatory agents, LPS and IFN-γ (FIGS. 4A and 4B). hMSCs pre-incubated with LPS or IFN-γ induced apoptosis of MDA cells in co-cultures (FIGS. 4C and 4D); however, pre-activated hMSCs with LPS induced more cell death because the up-regulation of TRAIL by LPS was 500-fold whereas the up-regulation by IFN-γ was only 30-fold. We also co-cultured activated hMSCs with other TRAIL sensitive cancer cell lines (FIG. 3D). The activated hMSCs were effective in reducing the live cell number in two triple negative breast cancer (TNBC) cell lines (HCC38 and MDA-MB-436) in addition to MDA, a pancreatic cancer cell line (CFPAC), a cervical cancer cell line (Hela), and carcinomic human alveolar basal epithelial cells (A549). Activated hMSCs had no effect on a line of glioblastoma cells (U87) even though MSCs transduced to express TRAIL were previously shown to inhibit intracranial U87 glioma growth (Menon et al., Stem Cells, Vol. 27, pgs. 2320-2330 (2009)). The discrepancy is explained probably by the observation that U87 cells are less sensitive to rhTRAIL than MDA cells (FIG. 4E). The results suggest that preconditioning hMSCs to express TRAIL can be useful, but gene modification may be necessary to obtain optimal therapeutic benefits in some circumstances.

We elected to focus on the three triple negative breast cancer cell lines. Induction of apoptosis by hMSCs in all three cell lines was reduced partially when TRAIL activity was inhibited by a TRAIL blocking antibody: MDA-MB-231 (denoted as MDA in FIGS. 3E and 3F), and HCC38 and MDA-MB-436 cells (FIG. 4F). The antibody was more effective in blocking the effects of rhTRAIL than in blocking the effects of pre-activated hMSCs (FIGS. 3E and 3F), apparently because the hMSCs were continually activated to express TRAIL in the co-culture system. Also, inhibition of a decoy receptor for TRAIL (osteoprotegerin: OPG) in hMSCs with troglitazone (Krause et al., Proc. Nat. Acad. Sci., Vol. 107, pgs. 4147-4152 (2010)) increased the apoptosis compared to control (FIG. 3G). The results indicated that activated hMSCs induced TRAIL-dependent apoptosis in the three triple negative breast cancer cell lines.

Conditions for Activation of hMSCs by TNF-α and Variations Among hMSC Preparations

The activation of hMSCs by TNF-α to induce apoptosis of MDA cells in co-culture was concentration dependent over the range of 0.1 to 10 ng/mL (FIG. 5A). Activation of hMSCs with as little as 0.1 ng/mL TNF-α was adequate to induce MDA apoptosis. Cell-to-cell contact was required, because the hMSCs had no effect in transwell co-cultures (FIG. 5B) and an increase in soluble TRAIL was not detected in conditioned medium from the cells (FIG. 6A), suggesting TRAIL expressed by hMSCs was transmembrane. The apoptosis of MDA cells in co-cultures increased with increasing ratios of hMSCs to MDA cells over the range of 0.06:1 to 2:1 (FIG. 5C). Control experiments demonstrated that human foreskin fibroblasts (Hs68) did not express TRAIL upon incubation with TNF-α (FIG. 6B) and they did not induce apoptosis of MDA cells upon co-culture (FIG. 6C). Two other samples of primary preparations of human dermal fibroblasts (hDF) slightly decreased the number of live MDA cells when co-cultured with the MDA cells (FIGS. 6D & 6E) and TNF-α, but the effect was not inhibited by a blocking anti-body to TRAIL (FIG. 6F).

As reported previously, there were variations in the quality of hMSCs obtained from bone marrow aspirates, even if the aspirates were drawn from the same normal volunteer at the same session and the hMSCs were isolated and expanded with a standardized protocol (Phinney et al., J. Cell. Biochem., Vol. 75, pgs. 424-436 (1999); Sekiya et al., Stem Cells, Vol. 20, pgs. 530-541 (2002); Wolfe et al. Methods Mol. Biol, Vol. 449, pgs 3-25, (2008)). Therefore, four preparations of hMSCs, identified by their anonymous donor numbers, were compared. The four samples of pre-activated hMSCs demonstrated large variations in the apoptosis induced in the MDA cells (FIG. 5D). As expected, the apoptosis induced by the hMSCs correlated with their levels of TRAIL expression following incubation with TNF-α (FIG. 5E).

As observed previously, cultures of hMSCs lose many of their biological properties as they are expanded beyond about 20 population doublings in culture (Digirolamo et al., Br. J. Haematol., Vol. 107, pgs. 275-281 (1999); Larson et al., Tissue Engineering Part A., Vol. 16, pgs. 3385-3394 (2010)). As expected, hMSCs gradually lost their ability to express TRAIL upon TNF-α activation (FIG. 5F) and to induce apoptosis of MDA cells as they were expanded through 20 or 25 population doublings (FIG. 5G). These observations demonstrated that apoptosis induced by TNF-α activated hMSCs required up-regulation of TRAIL and that the effectiveness of the cells varies with the quality of the hMSCs.

hMSCs Acted Synergistically with Doxorubicin in Suppressing MDA Cells

To examine synergistic interactions between TRAIL-expressing activated hMSCs and chemotherapeutic drugs, MDA cells were treated with both doxorubicin and hMSCs or activated hMSCs. As reported previously (Mallory et al., Mol. Pharmacol., Vol. 68, pgs. 1747-1756 (2005)), doxorubicin in a low concentration of 100 ng/mL (0.2 μM) suppressed proliferation of MDA as indicated by the decrease in recovery of live cells (FIG. 5I) but did not induce apoptosis (FIG. 5H). Incubation of MDA cells with doxorubicin decreased the number of live MDA cells recovered from cultures after 24 hrs. (FIG. 5I) in a dose dependent manner (FIG. 6G). Addition of hMSCs, however, together with 100 ng/mL doxorubicin further decreased the number of live MDA cells recovered from the cultures (FIG. 5I) and increased apoptosis greatly (FIG. 5H). The effect was synergistic in that the decrease in live MDA cells was greater than the additive effect observed with doxorubicin alone (FIG. 5H) and activated hMSCs alone (FIG. 3B). Of special note, the hMSCs were effective regardless of TNF-α activation (FIGS. 5H and 5I). Because doxorubicin enhances TRAIL-induced apoptosis by activating caspase or TRAIL receptors on cancer cells (Buchsbaum et al., Clin. Cancer Res., Vol. 9, pgs. 3731-3441 2003; Keane et al., Cancer Res., Vol. 59, pgs. 734-741 1999; Singh et al., Cancer Res., Vol. 63, pgs. 5390-5400 (2003)), the low level of TRAIL activation in hMSCs which was induced by the co-culture with MDA even without TNF-α (FIG. 6H) might be sufficient to induce the apoptosis in MDA cells and then these dead cells create feed-forward stimulation of TRAIL. This synergistic effect was replicated in two additional triple negative breast cancer cell lines HCC38 and MDA-MB-436 (FIG. 6I). Therefore, combination treatment of a chemotherapeutic drug and activated hMSCs can create synergistic effects and pre-activation of hMSCs with pro-inflammatory cytokines may not be essential to induce apoptosis in MDA cells exposed to doxorubicin.

Expression of TRAIL on hMSCs was Markedly Increased Upon Co-Culture

Apoptosis of MDA cells by activated hMSCs appeared to increase with time in culture (FIG. 7A). Therefore, the levels of TRAIL in hMSCs isolated from the co-cultures were assayed. There was a 10-fold increase in the expression of TRAIL in hMSCs recovered from co-cultures of MDA cells and activated hMSCs (FIGS. 7B and 7C). The results suggested that apoptotic MDA cells might enhance expression of TRAIL in hMSCs.

To test the hypothesis, hMSCs were incubated with apoptotic MDA cells. The apoptotic MDA cells were prepared by incubation with 100 ng/ml of recombinant human TRAIL (rhTRAIL) for 24 hrs in serum free media (FIG. 8A) and recovery of non-adherent cells from the cultures. As expected, apoptotic MDA cells enhanced TRAIL expression in TNF-α activated hMSCs to the same extent as in the co-culture system (compare FIG. 7D to FIG. 7B). The hypothesis that the effects of the apoptotic MDA cells were explained by RNA that is released from damaged tissue (Kariko et al., J. Biol. Chem., Vol. 279, pgs. 12542-12550 (2004)) then was tested. hMSCs were assayed for expression of TLR3, a specific receptor for RNA (Kariko et al., 2004) that increases NF-κB signaling and thereby triggers an essential step in the pathway for induction of TRAIL (Rivera-Walsh et al., J. Biol. Chem., Vol. 276, pgs. 40385-40388 (2001)). Expression of TLR3 in hMSCs was increased by incubation with TNF-α and further enhanced by co-culture of the activated hMSCs with MDA cells (FIG. 7E). Increased expression of TLR3 was also observed when hMSCs were treated with apoptotic MDA cells (FIG. 7F). Treatment of apoptotic MDA cells with RNase inhibited the increase of TRAIL in hMSCs (FIG. 7G). Treatment with DNase also inhibited the increase of TRAIL in hMSCs, however, the expression level of TLR9, a receptor for DNA (Zhang et al., Nature, Vol. 464, pgs. 104-107 (2010)), was low in hMSCs and was not up-regulated by treatment of TNF-α or apoptotic MDA cells (data not shown). The roles of RNA and TLR3 were confirmed by the observations that poly(I:C), a synthetic ligand for TLR3 (Alexopoulou et al., Nature, Vol. 413, pgs. 732-738, (2001)), increased expression of TRAIL in hMSCs (FIGS. 7H and 8B) and caused a small but statistically significant increase in MDA apoptosis when added to co-cultures (FIG. 7I). Furthermore, adding a TLR3 blocking antibody reduced apoptosis of MDA cells in the co-culture system and led to recovery of greater numbers of live MDA cells (FIGS. 7J and 8C). The results suggested that the further increase of TRAIL in hMSCs observed in co-cultures with MDA cells was mediated by feed-forward stimulation of TLR3 by RNA, by DNA, and probably by other DAMPs from apoptotic MDA cells.

Activation of hMSCs with TNF-α Inhibited Cell Cycle Progression in MDA Cells

In the co-culture system, pre-activated hMSCs also inhibited cell cycle progression in the recovered adherent viable MDA cells (FIGS. 9A and 10A). In transwell co-cultures, the inhibition was less: 3.3% increase in G1 (FIGS. 10B and 10C) versus 17.4% in co-cultures with direct contact between the cells (FIGS. 9A and 10A). The results therefore suggested that cell-to-cell contact was involved.

As surrogate markers of cell cycle arrest, expression of cyclin D1, cyclin D3 and p21 was assayed. The MDA cells from co-culture with activated hMSCs down-regulated expression of cyclin D1 and D3 (FIGS. 9B, 9C, and 9D) and up-regulated p21 expression (FIGS. 9E and 9F). TNF-α had no significant effect on the expression of cyclin D1, D3 and p21 in MDA cells (FIGS. 9B˜F). To test whether DKK3 up-regulation in activated hMSCs (FIGS. 1B, 11A, 11B and 11C) inhibited the Wnt/β-catenin mediated cell cycle progression in MDA cells (Tetsu and McCormick, Nature, Vol. 398, pgs. 422-426 (1999)), β-catenin was assessed by IF staining (FIGS. 11D and 11E). In control MDA cells, β-catenin was present either as discontinuous dot-like labeling in the cytoplasm or within the nucleus. In co-cultures, β-catenin was markedly decreased (FIGS. 11D and 11E). In addition, addition of rhDKK-3 decreased the number of β-catenin expressing MDA (FIGS. 11F and 12A) and proliferation of MDA cells (FIGS. 11G and 12B) were decreased by exogenous rhDKK-3 administration (FIGS. 11F, 12A, 11G, and 12B) without affecting the viability of MDA cells (FIG. 11H). As expected, decreasing expression of DKK-3 in hMSCs with siRNA (FIG. 12C) increased the recovery of live MDA cells from co-cultures (FIG. 11I) and the number of β-catenin positive cells (FIG. 11J). siRNA knockdown of DKK-3 had no effect on apoptosis in the co-culture system (FIG. 12D), indicating that expression of DKK-3 did not inhibit or promote TRAIL-induced apoptosis. Therefore, these data suggested that hMSC activated with TNF-α inhibited cell cycle progression in MDA cells by secreting DKK-3.

### DISCUSSION

The results obtained here demonstrated that hMSCs incubated with TNF-α expressed high levels of membrane-bound TRAIL and hMSCs pre-activated in culture to express TRAIL reduced the tumor burden in a xenograft mouse model of human breast cancer lung metastases. The results suggested therefore that appropriately pre-activated hMSCs may provide a useful therapeutic strategy for cancers. The majority of research involving hMSCs and metastatic cancers has been performed in lung metastasis models, because intravenously infused cells are likely to be entrapped in the lungs (Lee et al., Cell Stem Cell, Vol. 5, pgs. 54-63 (2009)). Therefore, the potential applications of the therapy may be limited to cancers of the lung; however, there are some indications that intravenously delivered hMSCs are able to incorporate into tumors outside of the lung (Ling et al., J. International Cancer Microenvironment Society, Vol. 3, pgs. 83-95 (2010) and that MSC homing is increased after radiation therapy (Klopp et al., Cancer Res., Vol. 67, pgs 11687-11695 (2007)). Whether intravenously infused activated MSCs are able to accumulate in sufficient numbers in tumors outside of the lung to slow their growth is yet to be determined. Direct injection into tumors, or intracardiac or arterial infusion may be more efficient.

In contrast to hMSCs, human fibroblasts failed to induce TRAIL-dependent apoptosis of MDA cells. In addition, the apoptosis of MDA cells appeared to increase with time in culture, an observation explained largely by a further increase in expression of TRAIL by hMSCs in co-culture. Most interestingly, the enhanced expression of TRAIL in hMSCs observed in co-cultures with MDA cells was mediated by feed-forward reaction that was accounted for in part by RNA released from apoptotic MDA cells interacting with TLR3 to increase NF-κB signaling and thereby activating a pathway for up-regulation of TRAIL (Rivera-Walsh et al., 2001). In addition, DNA from the apoptotic MDA cells may increase interactions of additional damage-associated molecular patterns (DAMPs) with hMSCs through different TLRs (Chen and Nunez, Nat. Rev. Immunol., Vol. 10, pgs. 826-837 (2010)) and enhance TRAIL expression, because treatment of the apoptotic MDA cells with DNase decreased their effectiveness in enhancing TRAIL expression in hMSCs. A recent study showed that microparticles released by tumor cells undergoing in vitro apoptosis contained both DNA and RNA, which can trigger responses via the Toll-like receptors (Reich and Pisetsky, Exp. Cell Res., Vol. 315, pgs. 760-768 (2009)). This observation may help explain the results that were obtained in the co-culture system. Also, although apoptotic cells are engulfed effectively by neighboring cells before releasing their intracellular content in vivo, a recent report indicated that macrophages often fail to engulf apoptotic cells until long after they have acquired the morphological hallmarks of apoptosis (Devitt et al., Cell Death and Differentation, Vol. 10, pgs. 371-382 (2003)). Therefore, the feed-forward reaction that increased expression of both TRAIL and TLR3 may have increased the effectiveness of the hMSCs in suppressing tumor progression in the mice even though the cells engrafted in the tumors for only a short period of time.

The results demonstrated that combination of hMSCs and a low concentration of doxorubicin, a chemotherapy drug commonly used for breast cancer, created a synergistic effect on apoptosis of MDA cells. Indeed, it was previously shown that doxorubicin synergistically enhances soluble recombinant protein TRAIL-mediated apoptosis by activating caspase or TRAIL receptors on cancer cells (Buchsbaum et al., 2003; Keane et al., 1999; Singh et al., 2003); however, the combination also increased toxicity in normal mammary epithelial cells (Keane et al., 1999). In contrast, the data showed that a low dose of doxorubicin combined with hMSCs was enough to induce synergistic effects on apoptosis in MDA cells, suggesting that this combination may be an effective therapy.

hMSCs from different preparations varied in their activation of TRAIL and efficacy in inducing apoptosis in MDA cells. The variations may reflect sampling problems in obtaining hMSCs with bone marrow aspirates or intrinsic differences in hMSCs from different donors (Phinney et al., J. Cell. Biochem., Vol. 75, pgs. 424-436 (1999)). In addition, the same preparations became less effective after they were expanded extensively in culture. Therefore, the observations may help to explain some of the conflicting results previously reported in the literature because relatively little attention was paid to the differences between rodent and human MSCs, the quality of MSC preparations and the conditions for expanding them in culture (Prockop et al., J. Cell. Mol. Med., Vol. 14, pgs 21 90-21 99 (2010)). Also, some of the conflicting results may be explained by the experiments being conducted with both TRAIL sensitive and TRAIL insensitive cancer cell lines.

In addition, it has been shown that IFN-α and IFN-β have anti-tumor effects against some cancer (Ida et al., Gann, Vol. 73, pgs. 952-960 (1982)) by inducing higher levels of TRAIL in immune cells, which displayed apoptotic activity on cancer cells (Arbour et al., Mult. Scler., Vol. 11, pgs. 562-657 (2005); Borden et al., J. Interferon Cytokine Res., Vol. 31, pgs. 433-440 (2011); Tecchio et al., Blood, Vol. 103, pgs. 3837-3844 (2004)). Interestingly, it has been shown recently that IFN-β treatment increased TRAIL in serum of patients with metastatic melanoma and the patient who had the sustained tumor regression showed the highest level of TRAIL (Borden et al., 2011). Therefore, we speculate that the variations in TRAIL expression in not only MSCs, but also cancer associated stromal cells or immune cells, also may reflect different susceptibilities to cancer metastases. TRAIL is accepted generally as not affecting non-cancer cells and few non-specific side effects have been reported with administration of MSCs. The results presented here, however, do not rule out the possibility that MSCs activated to express TRAIL may have non-specific side effects such as increasing the expression of non-oncogenes that may enhance the cancer cell growth (Luo et al., Cell, Vol. 136, pgs. 823-837 (2009)).

Furthermore, DKK-3 expressed by hMSCs inhibited cell cycle progression in MDA cells. This suppressive effect was enhanced by pre-activated hMSCs and reduced by a siRNA knock down of DKK-3 (FIG. 11I). In the co-culture system, there was a decrease in β-catenin and cell cycle proteins. These data suggested that DKK-3 expressed by hMSCs decreased cell cycle progression of MDA cells by suppressing Wnt/β-catenin-mediated signaling. Recently, inhibiting β-catenin signaling has been suggested as a potential treatment for cancer. Indeed, beneficial effects in colorectal cancer were observed by disrupting Wnt/β-catenin signaling with non-steroidal anti-inflammatory drugs (Castellone et al., Science, Vol. 310, pgs. 1504-1510 (2005); Shao et al., J. Biol. Chem., Vol. 280, pgs. 26565-26572 (2005)) or with natural antagonists of the Wnt pathway such as secreted frizzled-related proteins (Suzuki et al., Cancer Genet., Vol. 36, pgs. 417-422 (2004)), DKK (Gonzalez-Sancho et al., Oncogene, Vol. 24, pgs. 1098-1103 (2005)) or small molecules (Lepourcelet et al., Cancer Cell, Vol. 5, pgs. 91-102 (2004)). Also, evidence is accumulating that the secreted Wnt antagonist DKK-3 and its regulators may constitute effective therapeutic targets for most human cancers (Veeck and Dahl, Biochem. Biophys. Acta, Vol. 1825, pgs. 18-28 (2011)). Ectopic DKK-3 expression prevented nuclear accumulation of β-catenin and decreased the expression of the Wnt target genes c-Myc and cyclin-D1 in non-small cell lung cancer cell lines (Yue et al., Carcinogenesis, Vol. 29, pgs. 84-92 (2008)). The results here add new evidence that exogenous DKK-3 can inhibit Wnt/β-catenin mediated cell proliferation of cancer cells.

In summary, the data suggest that hMSCs activated to express TRAIL may have several advantages as therapy for some cancers: (a) they avoid the complexities and dangers encountered by viral transfection with the TRAIL gene; (b) they deliver the potent membrane tethered form of TRAIL and tumor suppressive protein DKK3 in high local concentrations to cancers; and (c) they may provide a therapy for metastatic cancers that may be effective if used in combination with chemotherapeutic drugs.

The disclosures of all patents, publications (including published patent applications), depository accession numbers, and database accession numbers are incorporated herein by reference to the same extent as if each patent, publication depository accession number, and database accession number were incorporated individually by reference.

It is to be understood, however, that the scope of the present invention is not to be limited to the specific embodiments described above. The invention may be practiced other than as particularly described and still be within the scope of the accompanying claims.

What is claimed is:

1. A method of treating a tumor in an animal, comprising:
administering to said animal mesenchymal stem cells which have been contacted with at least one agent that stimulates said mesenchymal stem cells to express increased amounts of at least one agent that inhibits, prevents, or destroys the growth of tumors, said mesenchymal stem cells being administered in an amount effective to treat said tumor in said animal.

2. The method of claim 1 wherein said at least one agent that stimulates said mesenchymal stem cells to express increased amounts of at least one agent that inhibits, prevents, or destroys the growth of tumor cells is TNF-α.

3. The method of claim 1 wherein said at least one agent that inhibits, prevents, or destroys the growth of tumor cells is selected from the group consisting of TRAIL and DKK-3.

4. The method of claim 1 wherein said tumor is a malignant tumor.

5. The method of claim 4 wherein said malignant tumor is breast cancer.

6. The method of claim 4 wherein said malignant tumor is lung cancer.

7. The method of claim 1 where said animal is a primate.

8. The method of claim 7 wherein said primate is a human.

9. A method of treating a tumor in an animal, comprising:
administering to said animal (a) mesenchymal stem cells which have been contacted with at least one agent that stimulates said mesenchymal stem cells to express increased amounts of at least one agent that inhibits, prevents, or destroys the growth of tumors; and
(b) at least one chemotherapeutic agent, wherein said mesenchymal stem cells and at least one chemotherapeutic agent are administered in amounts effective to treat said tumor in said animal.

10. The method of claim 9 wherein said at least one agent that stimulates said mesenchymal stem cells to express increased amounts of at least one agent that inhibits, prevents, or destroys the growth of tumor cells is TNF-α.

11. The method of claim 9 wherein said at least one agent that inhibits, prevents, or destroys the growth of tumor cells is selected from the group consisting of TRAIL and DKK-3.

12. The method of claim 9 wherein said tumor is a malignant tumor.

13. The method of claim 12 where said malignant tumor is breast cancer.

14. The method of claim 12 wherein said malignant tumor is lung cancer.

15. The method of claim 9 where said animal is a primate.

16. The method of claim 15 wherein said primate is a human.

17. The method of claim 9 wherein said at least one chemotherapeutic agent is doxorubicin.
